(19) United States
(12) Patent Application Publication
(10) Pub. No.: US 2020/0031784 A1
(43) Pub. Date: Jan. 30, 2020

(54) OZANIMOD ADDITION SALT CRYSTAL, PREPARATION METHOD, PHARMACEUTICAL COMPOSITION, AND USES

(71) Applicant: Solipharma LLC, Hagzhou, Zhejiang (CN)

(72) Inventors: Xiaohong SHENG, Hangzhou, Zhejiang (CN); Xiaoxia SHENG, Hangzhou, Zhejiang (CN); Jianfeng ZHENG, Hangzhou, Zhejiang (CN)

(21) Appl. No.: 16/603,416

(22) PCT Filed: Apr. 7, 2017
(86) PCT No.: PCT/CN2017/079654

§ 371 Date: Oct. 7, 2019

(51) Int. Cl. C07D 271/06

(52) U.S. Cl. CPC C07D 271/06; C07B 2200/13

(57) ABSTRACT

Disclosed is an ozanimod addition salt crystal, which is provided with one or more improved characteristics compared with a known ozanimod solid form. Also disclosed are a preparation method for the ozanimod addition salt crystal, a pharmaceutical composition of same, and uses thereof in preparing a medicament for diseases or disorders medically requiring optional regulation, activation, excitement, inhibition or antagonism of sphingosine-1-phosphate receptor.

### FIELD OF THE INVENTION

The present invention relates to the technical field of crystallization in pharmaceutical chemistry. Specifically, the present invention relates to ozanimod crystalline addition salt forms, its preparation methods, pharmaceutical compositions, and uses thereof.

### BACKGROUND

Ozanimod is a selective sphingosine-1-phosphate (SIP) receptor modulator, used for treatment for autoimmune diseases. Ozanimod shows strong data in pharmacokinetic, pharmacodynamic and safety in clinical trials, suitable for differentiated drug development strategies.

Ozanimod has the chemical name of 5-[3-[(1S)-2,3-dihydro-1-(2-hydroxyethylamino)-1H-indo1-4-yl]-1,2,4-oxadiazol-5-yl 2-isopropoxybenzonitrile, molecular formula of C₂₃H₂₄N₄O₃, molecular weight of 404.46, and CAS number of 1306760-87-1. The chemical structure is shown in the following formula (I).

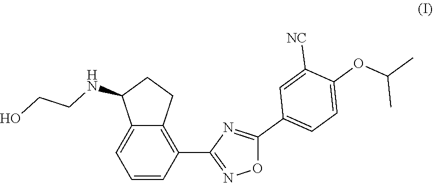 [SMILES: CC(C)Oc1ccc(-c2nc(-c3cccc4c3CC[C@@H]4NCCO)no2)cc1C#N]

Patent CN102762100B disclosed ozanimod and its hydrochloride, their preparation methods and their pharmaceutical compositions. The preparation of ozanimod hydrochloride required two steps: salt formation and recrystallization. The present inventor finds that the purity and yield of the ozanimod hydrochloride prepared according to the two steps are low, and the operation process is complex. It is difficult to remove impurities formed in the salt formation step by recrystallization, and the final product needs to be further purified to achieve higher purify.

The present inventor also found some other defects of ozanimod hydrochloride during research, such as low water solubility, apparent decrease in crystallinity in aqueous-organic solvent system and poor crystal phase stability.

In view of the disadvantages in the prior art, it is necessary to develop new crystalline ozanimod addition salts in order to meet the requirement for active ingredients in drug development.

### SUMMARY OF THE INVENTION

The present invention provides new crystalline forms of ozanimod addition salts, including ozanimod besylate Form 1, ozanimod citrate Form 1, ozanimod hemi-L-malate Form 1, ozanimod dihydrogen phosphate Form 1, ozanimod hydrosulfate Form 1, ozanimod hemi-sulfate Form 1, ozanimod L-tartrate Form 1, ozanimod hemi-fumarate Form 1, ozanimod fumarate Form 1, ozanimod maleate Form 1, ozanimod hydrobromide Form 1 and ozanimod mesylate Form 1.

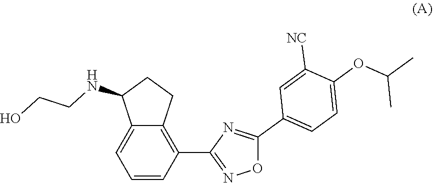 [SMILES: CC(C)Oc1ccc(-c2nc(-c3cccc4c3CC[C@@H]4NCCO)no2)cc1C#N]

Comparing to the known solid form of ozanimod hydrochloride in the prior art, the new crystalline ozanimod salt forms in the present invention have at least one or more advantageous properties, such as better solubility, lower hygroscopicity, higher crystallinity, faster dissolution rate, better crystal morphology, better phase stability, better storage stability, higher chemical purity and yield, better flowability and favorable processing characteristics.

One of the technical problems solved by the present invention is to provide crystalline ozanimod addition salt forms and their preparation methods.

The purpose is achieved by the following technical solutions.

The present invention provides crystalline forms of addition salts of ozanimod and the ozanimod is represented by the formula (A). The crystalline forms of addition salts are crystalline ozanimod mono-acid salt (the molar ratio of ozanimod and the corresponding acidic counter ion is 1:1) or crystalline ozanimod hemi-acid salt (the molar ratio of ozanimod and the corresponding acidic counter ion is 2:1). It is essentially a crystalline solid, preferably an anhydrate, a hydrate or a non-solvate.

The crystalline forms of the ozanimod addition salts include the following: ozanimod besylate Form 1, ozanimod citrate Form 1, ozanimod hemi L-malate Form 1, ozanimod dihydrogen phosphate Form 1, ozanimod hydrosulfate Form 1, ozanimod hemi-sulfate Form 1, ozanimod L-tartrate Form 1 ozanimod hemi-fumarate Form 1, ozanimod fumarate Form 1, ozanimod maleate salt Form 1, ozanimod hydrobromide Form 1, ozanimod mesylate Form 1.

In a preferred embodiment of the present invention, the ozanimod besylate of the present invention is the crystalline ozanimod besylate Form 1 (herein after referred to as “besylate Form 1”).

Using Cu—Kα radiation, the X-ray powder diffraction pattern of besylate Form 1, expressed as 2θ angles, has the following characteristic peaks: 5.7°±0.2°, 9.1°±0.2°, 13.9°±0.2° and 14.7°±0.2°.

More preferably, the X-ray powder diffraction pattern of the besylate Form 1, expressed as 2θ angles, further has one or more of the following characteristic peaks: 6.9°±0.2°, 11.4°±0.2, 18.8°±0.2° and 21.6°±0.2°.

Further preferably, the X-ray powder diffraction pattern of the besylate Form 1 also has one or more of the following characteristic peaks: 23.0°±0.2°, 23.3°±0.2°, 25.1°±0.2 ° and 26.3°±0.2°.

Non-restrictively, in one typical embodiment of the present invention, the X-ray powder diffraction pattern (XRPD) of the besylate Form 1 is shown in FIG. 2.

The Fourier transform infrared spectrum of the besylate Form 1 has characteristic peaks at wave numbers of 1612 cm⁻¹±2cm⁻¹, 1489 cm⁻¹±2 cm⁻¹, 1284 cm⁻¹±2 cm⁻¹, 1230 cm⁻¹±2cm⁻¹, 1158cm⁻¹±2 cm⁻¹, 1123 cm⁻¹±2 cm⁻¹, 1102 cm⁻¹±2 cm⁻¹, 1029 cm⁻¹±2 cm⁻¹, 1014 cm⁻¹±2 cm⁻¹, 759 cm⁻¹±2 cm⁻¹, 727 cm⁻¹±2 cm¹ and 614 cm⁻¹±2 cm⁻¹.

In a preferred embodiment of the present invention, the ozanimod citrate of the present invention is a crystalline ozanimod citrate Form 1 (herein after referred to as “citrate Form 1”).

Using Cu—Kα radiation, the X-ray powder diffraction pattern of citrate Form 1, expressed as 2θ angles, has the following characteristic peaks: 4.4°±0.2°, 14.0°±0.2°, 20.9°±0.2° and 24.9°±0.2°.

More preferably, the X-ray powder diffraction pattern of the citrate Form 1, expressed as 2θ angles, further has one or more of the following characteristic peaks: 12.5°±0.2°, 13.5°±0.2°, 14.3°±0.2° and 15.9°±0.2°.

Further preferably, the X-ray powder diffraction pattern of the citrate Form 1 also has one or more characteristic peaks: 20.6°±0.2°, 22.7°±0.2°, 24.5°±0.2° and 29.2°±0.2°.

Non-restrictively, in one typical embodiment of the present invention, the X-ray powder diffraction pattern(XRPD) of the citrate Form 1 is shown in FIG. 8.

The Fourier transform infrared spectrum of the citrate Form 1 has characteristic peaks at wave numbers of 1617 cm⁻¹±2 cm⁻¹, 1516 cm⁻¹±2 cm⁻¹, 1489 cm⁻¹±2 cm⁻¹, 1464 cm⁻¹±2 cm⁻¹, 1353 cm⁻¹±2 cm⁻¹, 1288 cm⁻¹±2 cm⁻¹, 1106 cm⁻¹±2cm⁻¹, 1079 cm⁻¹±2 cm⁻¹, 945 cm⁻¹±2 cm⁻¹, 837 cm⁻¹±2 cm⁻¹ and 762 cm⁻¹±2 cm⁻¹.

In a preferred embodiment of the present invention, the ozanimod hemi-L-malate of the present invention is the crystalline ozanimod hemi-L-malate salt Form 1 (herein after referred to as “hemi-L-malate Form 1”).

Using Cu—Kα radiation, the X-ray powder diffraction pattern of the hemi-L-malate Form 1, expressed as 2θ angles, has the following characteristic peak: 3.7°±0.2°, 14.8°±0.2°, 18.5°±0.2°, and 22.2°±0.2°.

More preferably, the X-ray powder diffraction pattern of the hemi-L-malate Form 1, expressed as 2θ angles, further has one or more of the following characteristic peaks: 7.3°±0.2°, 12.0°±0.2, 24.5°±0.2 ° and 26.0°±0.2°.

Further preferably, the X-ray powder diffraction pattern of the hemi-L-malate Form 1 also has one or more of the following characteristic peaks: 12.6°±0.2°, 13.9°±0.2°, 19.7°±0.2° and 20.1°±0.2°.

Non-restrictively, in one typical embodiment of the present invention, the XRPD pattern of the hemi-L-malate Form 1 is shown in FIG. 14.

The Fourier transform infrared spectrum of the hemi-L-malate Form 1 has characteristic peaks at wave numbers of 1710 cm⁻¹±2 cm⁻¹, 1618 cm⁻¹±2 cm⁻¹, 1496 cm⁻¹±2 cm ¹, 1354 cm⁻¹±2 cm⁻¹, 1284 cm⁻¹±2 cm⁻¹, 1100 cm⁻¹±2 cm⁻¹, 942 cm⁻¹±2 cm⁻¹, 833 cm⁻¹±2 cm⁻¹, 758 cm⁻¹±2 cm⁻¹ and 663 cm⁻¹±2 cm⁻¹.

In a preferred embodiment of the present invention, the ozanimod dihydrogen phosphate of the present invention is the crystalline ozanimod dihydrogen phosphate Form 1 (herein after referred to as “dihydrogen phosphate Form 1”).

Using Cu—Kα radiation, the X-ray powder diffraction pattern of the dihydrogen phosphate Form 1, expressed as 2θ angles, has the following characteristic peak: 3.3°±0.2°, 5.5°±0.2°, 11.2°±0.2° and 20.8°±0.2°.

More preferably, the X-ray powder diffraction pattern of the dihydrogen phosphate salt Form 1, expressed as 2θ angles, further has one or more of the following characteristic peaks: 3.6°±0.2°, 7.4°±0.2°, 13.1°±0.2° and 22.7°±0.2°.

Further preferably, the X-ray powder diffraction pattern of the dihydrogen phosphate Form 1, also has one or more of the following characteristic peaks: 13.8°±0.2°, 17.0°±0.2°, 24.3°±0.2°, and 28.9°±0.2°.

Non-restrictively, in one typical embodiment of the present invention, the X-ray powder diffraction pattern (XRPD) of the dihydrogen phosphate Form 1 is shown in FIG. 20.

The Fourier transform infrared spectrum of the dihydrogen phosphate Form 1 has characteristic peaks at wave numbers of 1618 cm⁻¹±2 cm⁻¹, 1490 cm⁻¹±2 cm⁻¹, 1464 cm⁻¹±2 cm⁻¹, 1354 cm⁻¹±2 cm⁻¹, 1288 cm⁻¹±2 cm⁻¹, 1103 cm⁻¹±2 cm⁻¹, 1006 cm⁻¹±2 cm⁻, 957 cm⁻¹±2 cm⁻¹, 835 cm⁻¹±2 cm⁻¹ and 762 cm⁻¹±2 cm⁻¹.

In a preferred embodiment of the present invention, the ozanimod hydrosulfate of the present invention is the crystalline ozanimod hydrosulfate Form 1 (herein after referred to as hydrosulfate Form 1″).

Using Cu—Kα radiation, the X-ray powder diffraction pattern of the hydrosulfate Form 1, expressed as 2θ angles, has one or more of the following characteristic peaks: 4.1°±0.2°, 8.3°±0.2°, 11.1°±0.2°, and 16.8°±0.2°.

More preferably, the X-ray powder diffraction pattern of the hydrosulfate Form 1, expressed as 2θ angles, further has one or more of the following characteristic peaks: 14.6°±0.2 °, 18.5°±0.2°, 21.3°±0.2°, and 22.8 20 ±0.2°.

Further preferably, the X-ray powder diffraction pattern of the hydrosulfate Form 1, also has one or more of the following characteristic peaks: 17.0°±0.2°, 22.4 °±0.2°, 24.7°±0.2° , and 25.8°±0.2°.

Non-restrictively, in one typical embodiment of the present invention, the X-ray powder diffraction pattern (XRPD) of the hydrosulfate Form 1 is shown in FIG. 26.

The Fourier transform infrared spectrum of the hydrosulfate Form 1 has characteristic peaks at wave numbers of 1614 cm⁻¹±2 cm⁻¹, 1488 cm⁻¹±2 cm⁻¹, 1461 cm⁻¹±2 cm⁻¹, 1287 cm⁻¹±2 cm and 1179 cm ⁻¹±2 cm⁻¹, 1155 cm⁻¹±2 cm⁻¹, 1051 cm⁻¹±2 cm⁻¹, 867 cm⁻¹±2 cm⁻¹ and 759 cm⁻¹±2 cm⁻¹.

In a preferred embodiment, the ozanimod hemi-sulfate of the present invention is the crystalline ozanimod hemi-sulfate Form 1 (herein after referred to as “hemi-sulfate Form 1”).

Using Cu—Kα radiation, the X-ray powder diffraction pattern of the hemi-sulfate Form 1, expressed as 2θ angles, has the following characteristic peaks: 3.8°±0.2°, 11.6°±0.2°, 13.3°±0.2, and 19.5°±0.2°.

More preferably, the X-ray powder diffraction pattern of the hemi-sulfate Form 1, expressed as 2θ angles, further has one or more of the following characteristic peaks: 9.9°±0.2°, 15.3°±0.2°, 22.1°±0.2°, and 24.6°±0.2°.

Further preferably, the X-ray powder diffraction pattern of the hemi-sulfate Form 1, also has one or more of the following characteristic peaks: 15.7°±0.2°, 20.1°±0.2°, 25.3°±0.2°, and 27.9°±0.2°.

Non-restrictively, in one typical embodiment of the present invention, the X-ray powder diffraction pattern (XRPD) of the hemi-sulfate Form 1 is shown in FIG. 32.

The Fourier transform infrared spectrum of the hemi-sulfate Form 1 has characteristic peaks at wave numbers of 1620 cm⁻¹±2 cm⁻¹, 1462 cm⁻¹±2 cm⁻¹, 1406 cm⁻¹±2 cm⁻¹, 1284 cm⁻¹±2 cm⁻¹, 1128 cm⁻¹±2 cm⁻¹, 1090 cm⁻¹±2 cm⁻¹, 1041 cm⁻¹±2 cm⁻¹, 1013 cm⁻¹±2 cm⁻¹, 941 cm⁻¹±2 cm⁻¹, 838 cm⁻¹±2 cm⁻¹ and 761 cm⁻¹±2 cm⁻¹.

In a preferred embodiment of the present invention, the ozanimod L-tartrate of the present invention is the crystalline ozanimod L-tartrate Form 1 (herein after referred to as “L-tartrate salt Form 1”).

Using Cu—Kα radiation, the X-ray powder diffraction pattern of the L-tartrate Form 1, expressed as 2θ angles, has the following characteristic peaks: 6.4°±0.2°, 9.9°±0.2°, 12.7°±0.2°, and 22.8°±0.2°.

More preferably, the X-ray powder diffraction pattern of the L-tartrate Form 1, expressed as 2θ angles, further has one or more of the following characteristic peaks: 3.1°+0.2°, 5.5°±0.2°, 10.6°±0.2° and 14.8°±0.2°.

Further preferably, the X-ray powder diffraction pattern of the L-tartrate Form 1, also has one or more of the following characteristic peaks: 7.0°±0.2°, 13.0°±0.2°, 16.6°±0.2°, and 19.0°±0.2°.

Non-restrictively, in one typical embodiment of the present invention, the X-ray powder diffraction pattern (XRPD) of the L-tartrat Form 1 is shown in FIG. 38.

The Fourier transform infrared spectrum of the L-tartrat salt Form 1 has characteristic peaks at wave numbers of 1610 cm⁻¹±2 cm⁻¹, 1569 cm⁻¹±2 cm⁻¹, 1486 cm⁻¹±2 cm⁻¹, 1460 cm⁻¹±2 cm⁻¹, 1362 cm⁻¹±2 cm⁻¹, 1280 cm⁻¹±2 cm⁻¹, 1155 cm⁻¹±2 cm⁻¹, 1104 cm⁻¹±2 cm⁻¹, 1061 cm⁻¹±2 cm⁻¹, 942 cm⁻¹±2 cm⁻¹ and 759 cm⁻¹±2 cm⁻¹.

In a preferred embodiment of the present invention, the ozanimod hemi-fumarate of the present invention is the crystalline ozanimod hemi-fumarate Form 1 (herein after referred to as “hemi-fumarate Form 1”).

Using Cu—Kα radiation, the X-ray powder diffraction pattern of the hemi-fumarate

Form 1, expressed as 2θ angles, further has the following characteristic peaks: 6.3°±0.2°, 9.0°±0.2, 12.6°±0.2, and 13.7°±0.2°.

More preferably, the X-ray powder diffraction pattern of the hemi-fumarate Form 1, expressed in terms of 2θ angle, also has one or more of the following characteristic peaks: 12.9°±0.2°, 14.5°±0.2°, 17.3 °±0.2° and 21.5°±0.2°.

More preferably, the X-ray powder diffraction pattern of the hemi-fumarate Form 1, expressed as 2θ angles, has the following one or more characteristic peaks: 8.6°±0.2°, 21.0°±0.2°, 22.8°±0.2, and 25.7±0.2°.

Non-restrictively, in one typical embodiment of the present invention, the X-ray powder diffraction pattern (XRPD) of the hemi-fumarate Form 1 is shown in FIG. 44.

The Fourier transform infrared spectrum of the hemi-fumarate Form 1 has characteristic peaks at wave numbers of 1615 cm⁻¹±2 cm⁻¹, 1576 cm⁻¹±2 cm⁻¹, 1493 cm⁻¹±2cm⁻¹, 1405 cm⁻¹±2 cm⁻¹, 1351 cm⁻¹±2 cm⁻¹, 1284 cm⁻¹±2 cm⁻¹, 1099 cm⁻¹±2 cm⁻¹, 944 cm⁻¹±2 cm⁻¹, 833 cm⁻¹±2 cm⁻¹, 760 cm⁻¹±2 cm⁻¹ and 652 cm⁻¹±2 cm⁻¹.

In a preferred embodiment of the present invention, the ozanimod fumarate of the present invention is the crystalline ozanimod fumarate Form 1 (herein after referred to as “fumarate Form 1”).

Using Cu—Kα radiation, the X-ray powder diffraction pattern of the fumarate Form 1, expressed as 2θ angles, has the following characteristic peaks: 3.9°±0.2°, 7.9°±0.2°, 13.3°±0.2°, and 17.0°±0.2°.

More preferably, the X-ray powder diffraction pattern of the fumarate Form 1, expressed as 2θ angles, further has one or more of the following characteristic peaks: 7.5°±0.2°, 15.8°±0.2, 24.6°±0.2° and 28.6°±0.2°.

Further preferably, the X-ray powder diffraction pattern of the fumarate Form 1, also has one or more of the following characteristic peaks: 13.8°±0.2°, 20.1°±0.2°, 23.3°±0.2°, and 23.8°±0.2°.

Non-restrictively, in one typical embodiment of the present invention, the X-ray powder diffraction pattern (XRPD) of the fumarate Form 1 is shown in FIG. 50.

The Fourier transform infrared spectrum of the fumarate Form 1 has characteristic peaks at wave numbers of 1701 cm⁻¹±2 cm⁻¹, 1614 cm⁻¹±2 cm⁻¹, 1484 cm⁻¹±2 cm⁻¹, 1462 cm⁻¹±2 cm⁻¹, 1342 cm⁻¹±2 cm⁻¹, 1284 cm⁻¹±2 cm⁻¹, 1103 cm⁻¹±2 cm⁻¹, 986 cm⁻¹±2 cm⁻¹, 759 cm⁻±2 cm⁻¹ and 639 cm⁻¹±2 cm⁻¹.

In a preferred embodiment of the present invention, the ozanimod maleate salt of the present invention is the crystalline ozanimod maleate Form 1 (herein after referred to as “maleate salt Form 1”).

Using Cu—Kα radiation, the X-ray powder diffraction pattern of the maleate Form 1, expressed as 2θ angles, has the following characteristic peaks: 8.2°±0.2°, 11.5°±0.2°, 12.4°±0.2°, and 13.6°±0.2°.

More preferably, the X-ray powder diffraction pattern of the maleate salt Form 1, expressed as 2θ angles, further has the one or more of the following characteristic peaks: 5.3°±0.2°, 6.7°±0.2, 10.2°±0.2° and 11.0°±0.2°.

Further preferably, the X-ray powder diffraction pattern of the maleate salt Form 1, also has one or more of the following characteristic peaks: 14.1°±0.2°, 15.8°±0.2°, 16.4°±0.2°, and 18.1°±0.2°.

Non-restrictively, in one typical embodiment of the present invention, the X-ray powder diffraction pattern (XRPD) of the maleate salt Form 1 is shown in FIG. 56.

The Fourier transform infrared spectrum of the maleate Form 1 has characteristic peaks at wave numbers of 1700 cm⁻¹±2 cm⁻¹, 161 cm⁻¹±2 cm⁻¹, 1487 cm⁻¹±2 cm⁻¹, 1461 cm⁻¹±2 cm⁻¹, 1353 cm⁻¹±2 cm⁻¹, 1281 cm⁻¹±2 cm⁻¹, 1102 cm⁻¹±2 cm⁻¹, 1087 cm⁻¹±2 cm⁻¹, 865 cm⁻¹±2 cm⁻¹, 759 cm⁻¹±2 cm⁻¹ and 653 cm⁻¹±2 cm⁻¹.

In a preferred embodiment of the present invention, the ozanimod hydrobromide of the present invention is the crystalline ozanimod hydrobromide salt Form 1 (herein after referred to as “hydrobromide salt Form 1”).

Using Cu—Kα radiation, the X-ray powder diffraction pattern of the hydrobromide Form 1, expressed as 2θ angles, has the following characteristic peaks: 3.9°±0.2°, 12.1°±0.2°, 13.7°±0.2° and 20.3°±0.2°.

More preferably, the X-ray powder diffraction pattern of the hydrobromide Form 1, expressed as 2θ angles, further has the following one or more characteristic peaks: 12.9°±0.2°, 22.7°±0.2°, 24.5°±0.2° and 26.2°±0.2°.

Further preferably, the X-ray powder diffraction pattern of the hydrobromide salt Form 1, also has one or more of the following characteristic peaks: 12.4°±0.2°, 19.5°±0.2°, 21.3°±0.2° and 26.8°±0.2°.

Non-restrictively, in one typical embodiment of the present invention, the X-ray powder diffraction pattern (XRPD) of the hydrobromide Form 1 is shown in FIG. 62.

The Fourier transform infrared spectrum of the hydrobromide Form 1 has characteristic peaks at wave numbers of 3276 cm⁻¹±2 cm⁻¹, 1620 cm⁻¹±2 cm⁻¹, 1498 cm⁻¹±2 cm⁻¹, 1443 cm⁻¹±2 cm⁻¹, 1405 cm⁻¹±2 cm⁻¹, 1353 cm⁻¹±2 cm⁻¹, 1285 cm⁻¹±2 cm⁻¹, 1099 cm⁻¹±2cm⁻¹, 1074 cm⁻¹±2 cm⁻¹, 942 cm⁻¹±2 cm⁻¹, 837 cm⁻¹±2cm⁻¹ and 761 cm⁻¹±2 cm⁻¹.

In a preferred embodiment of the present invention, the ozanimod mesylate of the present invention is the crystalline ozanimod mesylate Form 1 (herein after referred to as “mesylate Form 1”).

Using Cu—Kα radiation, the X-ray powder diffraction pattern of the mesylate salt Form 1, expressed as 2θ angles, has the following characteristic peaks: 11.6°±0.2°, 12.6°±0.2°, 18.2°±0.2° and19.5°±0.2°.

More preferably, the X-ray powder diffraction pattern of the mesylate Form 1, expressed as 2θ angles, has one or more of the following characteristic peaks: 4.9°±0.2°, 7.9°±0.2°, 9.9°±0.2° and 16.8°±0.2°.

Further preferably, the X-ray powder diffraction pattern of the mesylate Form 1, also has one or more of the following characteristic peaks: 20.1°±0.2°, 23.1°±0.2°, 23.4°±0.2°, 24.3°±0.2° and 25.0°±0.2°.

Non-restrictively, in one typical embodiment of the present invention, the X-ray powder diffraction pattern (XRPD) of the mesylate Form 1 is shown in FIG. 68.

The Fourier transform infrared spectrum of the mesylate Form 1 has characteristic peaks at wave numbers of 1617 cm⁻¹±2 cm⁻¹, 1492 cm⁻¹±2 cm⁻¹, 1406 cm⁻¹±2 cm⁻¹, 1357 cm⁻¹±2 cm⁻¹, 1285 cm⁻¹±2 cm⁻¹, 1152 cm⁻¹±2 cm⁻¹, 1105 cm⁻¹±2 cm⁻¹, 1044 cm⁻¹±2 cm⁻¹, 940cm⁻¹±2 cm⁻¹, 780 cm⁻¹±2 cm⁻¹ and 760 cm⁻¹±2 cm⁻¹.

The preparation of the crystalline forms of the ozanimod addition salt comprises the following steps.

Forming an ozanimod solution and the corresponding acid solution respectively in a co-solvent, mixing both solutions, and then preparing the crystalline forms using the following method I or method II.

Method I: stirring the mixture solution, separating and drying the precipitated crystals to obtained the crystalline ozanimod mono-acid addition salt forms or crystalline ozanimod hemi-acid addition salt forms.

Method II: adding an anti-solvent to the mixture solution, stirring it, separating and drying the precipitated crystals to obtained crystalline ozanimod mono-acid addition salt forms or crystalline ozanimod hemi-acid addition salt forms.

In method I or method II, the co-solvent is selected from the group consisting of an alcohol, a ketone, and any mixture thereof

Preferably, in method I or method II, the co-solvent is selected from the group consisting of a C₁ to C₄ alcohol, a C₃ to C₄ ketone, and any mixture thereof, more preferably selected from the group consisting of propanol, acetone, and any mixture thereof

Preferably, in the method I, the concentration of ozanimod in the co-solvent is 0.5 to 1.05 times of the solubility of ozanimod in the selected solvent.

Preferably, in the method II, the concentration of ozanimod in the co-solvent is 0.1 to 1.05 times of the solubility of ozanimod in the selected solvent, more preferably 0.1 to 0.4 times.

Preferably, in method II, the anti-solvent is selected from the group consisting of an ester, an ether, an alkane, and any mixture thereof, more preferably selected from the group consisting of ethyl acetate, methyl tert-butyl ether, n-heptane, and any mixture thereof;

Preferably, in the preparation of an ozanimod mono-acid salt, the molar ratio of ozanimod and the acid counter ion is from 1:1.0 to 1:1.5, more preferably from 1:1.0 to 1:1.2.

Preferably, in the preparation of an ozanimod hemi-acid salt, the molar ratio of ozanimod and the acid counter ion is from 1:0.5 to 1:0.8, more preferably from 1:0.5 to 1:0.6.

Preferably, the stirring time is from 1 to 7 days, more preferably from 3 to 7 days.

Preferably, the operation temperature of the preparation method is from 10° C. to 40° C., more preferably room temperature.

Preferably, the drying temperature is room temperature, and the drying time is from 16 to 48 hours.

Compared with the ozanimod hydrochloride in prior art, the ozanimod besylate Form 1 of the present invention has the following beneficial properties:

1) According to the XRPD, the ozanimod besylate Form 1 is an excellent crystalline solid.

2) According to the isothermal adsorption curve, the weight changes in the range of 0% to 50% for the ozanimod besylate Form 1 and ozanimod hydrochloride are 0.05% and 0.14% respectively. The ozanimod besylate Form 1 in the present invention is less hygroscopic.

3) According to Comparative Example 2, the ozanimod besylate Form 1 has better phase stability than the ozanimod hydrochloride in aqueous-organic solvents.

4) According to Comparative Example 3, the ozanimod besylate Form 1 has higher chemical purity and yield than the ozanimod hydrochloride.

Compared with the ozanimod hydrochloride in the prior art, the ozanimod citrate Form 1 of the present invention has the following beneficial properties:

1) According to the XRPD pattern, the ozanimod citrate Form 1 is an excellent crystalline solid.

2) According to Comparative Example 2, the ozanimod citrate Form 1 has better phase stability than the ozanimod hydrochloride in aqueous-organic solvents.

3) According to Comparative Example 3, the ozanimod citrate Form 1 has higher chemical purity and yield than the ozanimod hydrochloride in aqueous organic solvents.

Compared with the ozanimod hydrochloride in the prior art, the ozanimod hemi-L-malate Form 1 of the present invention has the following beneficial properties:

1) According to the XRPD pattern, the ozanimod hemi-L-malate Form 1 is an excellent crystalline solid.

2) According to Comparative Example 2, the ozanimod hemi-L-malate Form 1 has better phase stability than the ozanimod hydrochloride in aqueous-organic solvents.

3) According to Comparative Example 1, the ozanimod hemi-L-malate Form 1 has better water solubility than the ozanimod hydrochloride.

4) According to Comparative Example 3, the ozanimod hemi-L-malate Form 1 has higher chemical purity and yield than the ozanimod hydrochloride in aqueous-organic solvents.

Compared with the ozanimod hydrochloride in prior art, the ozanimod dihydrogen phosphate Form 1 of the present invention has the following beneficial properties:

1) According to the XRPD pattern, the ozanimod dihydrogen phosphate Form 1 is an excellent crystalline solid.

2) According to Comparative Example 2, the ozanimod dihydrogen phosphate Form 1 has better phase stability than the ozanimod hydrochloride in aqueous-organic solvents.

3) According to Comparative Example 1, the ozanimod dihydrogen phosphate Form 1 has better water solubility than the ozanimod hydrochloride.

4) According to Comparative Example 3, the ozanimod dihydrogen phosphate Form 1 has higher chemical purity and yield than the ozanimod hydrochloride in aqueous-organic solvents.

Compared with the ozanimod hydrochloride in the prior art, the ozanimod hydrosulfate Form 1 of the present invention has the following beneficial properties:

1) According to the XRPD pattern, the ozanimod hydrosulfate Form 1 is an excellent crystalline solid.

2) According to Comparative Example 2, the ozanimod hydrosulfate Form 1 has better phase stability than the ozanimod hydrochloride in aqueous-organic solvents.

3) According to Comparative Example 3, the ozanimod hydrosulfate Form 1 has higher chemical purity and yield than the ozanimod hydrochloride in aqueous-organic solvents.

Compared with the ozanimod hydrochloride in the prior art, the ozanimod hemi-sulfate Form 1 of the present invention has the following beneficial properties:

1) According to the XRPD pattern, the ozanimod hemi-sulfate Form 1 is an excellent crystalline solid.

2) According to Comparative Example 2, the ozanimod hemi-sulfate Form 1 has better phase stability than the ozanimod hydrochloride in aqueous-organic solvents.

3) According to Comparative Example 3, the ozanimod hemi-sulfate Form 1 has higher chemical purity and yield than the ozanimod hydrochloride.

Compared with the ozanimod hydrochloride in the prior art, the ozanimod L-tartrate Form 1 of the present invention has the following beneficial properties:

1) According to the XRPD pattern, the ozanimod L-tartrate Form 1 is an excellent crystalline solid.

2) According to Comparative Example 2, the ozanimod L-tartrate Form 1 has better phase stability than the ozanimod hydrochloride in aqueous-organic solvents.

3) According to Comparative Example 3, the ozanimod L-tartrate Form 1 has higher chemical purity and yield than the ozanimod hydrochloride.

Compared with the ozanimod hydrochloride in the prior art, the ozanimod hemi-fumarate Form 1 of the present invention has the following beneficial properties:

1) According to the XRPD pattern, the ozanimod hemi-fumarate Form 1 is an excellent crystalline solid.

2) According to comparative example 2, the ozanimod hemi-fumarate Form 1 has better phase stability than the ozanimod hydrochloride in aqueous organic solvents.

3) According to comparative example 3, the ozanimod hemi-fumarate Form 1 has higher chemical purity and yield than the ozanimod hydrochloride.

Compared with the ozanimod hydrochloride in the prior art, the ozanimod fumarate Form 1 of the present invention has the following beneficial properties:

1) According to the XRPD pattern, the ozanimod fumarate Form 1 is an excellent crystalline solid.

2) According to comparative example 2, the ozanimod fumarate Form 1 has better phase stability than the ozanimod hydrochloride in aqueous-organic solvents.

3) According to Comparative Example 1, the ozanimod fumarate Form 1 has better water solubility than the ozanimod hydrochloride.

4) According to Comparative Example 3, the ozanimod fumarate Form 1 has higher chemical purity and yield than the ozanimod hydrochloride.

Compared with the ozanimod hydrochloride in the prior art, the ozanimod maleate

Form 1 of the present invention has the following beneficial properties: 1) According to the XRPD pattern, the ozanimod maleate Form 1 is an excellent crystalline solid.

2) According to comparative example 2, the ozanimod maleate Form 1 has better phase stability than the ozanimod hydrochloride in aqueous organic solvents.

3) According to Comparative Example 1, the ozanimod maleate Form 1 has better water solubility than the ozanimod hydrochloride.

4) According to Comparative Example 3, the ozanimod maleate Form 1 has higher chemical purity and yield than the ozanimod hydrochloride.

Compared with the ozanimod hydrochloride in the prior art, the ozanimod hydrobromide Form 1 of the present invention has the following beneficial properties:

1) According to the XRPD pattern, the ozanimod hydrobromide Form 1 is an excellent crystalline solid.

2) According to Comparative Example 2, the ozanimod hydrobromide Form 1 has better phase stability than the ozanimod hydrochloride in aqueous-organic solvents.

3) According to Comparative Example 1, the ozanimod hydrobromide Form 1 has better water solubility than the ozanimod hydrochloride.

4) According to Comparative Example 3, the ozanimod hydrobromide Form 1 has higher chemical purity and yield than the ozanimod hydrochloride.

Compared with the ozanimod hydrochloride in the prior art, the ozanimod mesylate Form 1 of the present invention has the following beneficial properties:

1) According to the XRPD pattern, the ozanimod mesylate Form 1 is an excellent crystalline solid.

2) According to comparative example 2, the ozanimod mesylate Form 1 has better phase stability than the ozanimod hydrochloride in aqueous-organic solvents.

3) According to Comparative Example 1, the ozanimod mesylate Form 1 has better water solubility than the ozanimod hydrochloride.

4) According to Comparative Example 3, the ozanimod mesylate Form 1 has higher chemical purity and yield than the ozanimod hydrochloride.

The above beneficial properties of crystalline ozanimod addition salt forms indicate that: compared to the known solid form of ozanimod hydrochloride in the prior art, the crystalline ozanimod addition salt forms of the present invention have at least one or more beneficial properties, such as crystallinity, hygroscopicity, water solubility, phase stability, chemical purity and yield, etc., and are more suitable to be the active ingredient of pharmaceutic formulation.

Crystalline solids have better flowability and processability for formulation (for example: filtration, drying, weighing, screening and other operations in the process of pharmaceutical preparation), and are advantageous to improve the homogeneity of the active ingredients and formulation.

Lower hygroscopicity, better phase stability and higher chemical purity can better ensure the potency of crystalline ozanimod addition salt forms themselves and the pharmaceutical formulation containing crystalline ozanimod addition salt forms, avoiding or reducing quality, safety and stability problems in the manufacturing process and/or storage, such as inhomogeneity, impurities and so on, and avoiding the special and expensive packaging. High preparation yield can significantly reduce production costs and increase production efficiency.

In any preparation methods of ozanimod addition salts and their forms in the present invention:

Unless otherwise specified, the “room temperature” refers to 10 to 30° C.

“Stirring” may be carried out by a conventional method in the art, such as magnetic stirring, mechanical stirring, and the stirring speed is 50 to 1800 rpm, preferably 300 to 900 rpm.

“Separation” may be performed using conventional methods in the field, such as concentration, volatilization, centrifugation or filtration under reduced pressure and pressure less than 0.09MPa is preferred. Preferably, pressure less than 0.09 MPa.

“Drying” may be performed by routine methods in the field, such as room temperature drying, forced air drying or vacuum drying. Drying is performed under reduced pressure or atmospheric pressure, and pressure less than 0.09MPa is preferred. Drying instruments and methods are unrestricted, and may be fume hood, blast oven, spray drying, fluidized bed drying or vacuum oven. Preferably, pressure less than 0.09 MPa.

In the present invention, the “crystal” or “crystal form” or “crystalline form” refers to that characterized by X-ray powder diffraction pattern, having a unique ordered molecular arrangement or configuration within the crystalline lattice. It is known to those skilled in the field that the experimental error depends on instrumental conditions, sample preparation and sample purity. The 2θ angle of the peaks in the XRPD pattern may change with the change of instrument and samples. The difference of peak position may vary by 1°, 0.8°, 0.5°, 0.3°, 0.1°, etc., and ±0.2° is usually allowed. The relative intensity of peaks may change with the change of sample, sample preparation, and other experimental conditions. Therefore, the order of peak intensities should not be regarded as the only or the determining factor. Due to the effect of experimental factors including sample height, peak position may shift. Generally, a small amount of peak shifting is acceptable. Hence, it is easily understood for those skilled in the field that any crystal form having the same or similar X-ray powder diffraction pattern as that of the crystal form in the present invention should be within the scope of the present invention. “Single crystal form” refers to a crystal form confirmed by X-ray powder diffraction as a single form.

In the present invention, the crystalline forms of ozanimod addition salts are substantially pure, single, or substantially free of any other crystalline or amorphous forms. As used herein, “substantially pure” when used in reference to a new crystalline form means that the new crystalline form comprises at least 80% by weight of the present compound, more preferably at least 90% (by weight), especially at least 95% (by weight), especially at least 99% (by weight).

Furthermore, the second technical problem solved in the present invention is to provide a pharmaceutical composition, which comprises a therapeutically and/or prophylactically effective amount of one or more ozanimod addition salts or crystalline forms of ozanimod addition salts in the present invention, and at least one pharmaceutically acceptable carrier. In addition, the pharmaceutical composition may further comprise other pharmaceutically acceptable ozanimod crystalline forms, amorphous ozanimod or other ozanimod salts.

The excipients of pharmaceutical composition are known to those skilled in the field, and the selection of the type, usage and amount of the excipients is also known to those skilled in the field. They include carbohydrate, cellulose and its derivative, starch or modified starch, solid inorganics such as calcium phosphate, dicalcium phosphate, hydroxyapatite, calcium sulfate, calcium carbonate, semi-solid such as lipids or paraffin wax, adhesive such as microcrystalline cellulose, ethyl cellulose, hydroxymethyl cellulose, hydroxypropyl methyl cellulose, hydroxyethyl cellulose, glidants such as colloidal silica dioxide, light anhydrous silicic acid, crystalline cellulose, talcum powder or magnesium stearate, disintegrants such assodium glycolate starch, crospovidone, croscarmellose, sodium carboxymethylcellulose, cornstarch, lubricant such as stearic acid, magnesium stearate, sodium stearyl fumarate, polyethyleneglycol.

The administrative routes of pharmaceutical compositions may be oral, intravenous injection, injection into tissue, transdermal, rectal, nasal dripping, etc. The pharmaceutical compositions may be made in certain dosage form depending on the administration routes or needs, such as in solid or liquid form. Solid oral dosage forms, such as tablets, granules, powders, pills and capsules; liquid oral dosage forms, such as solution, syrups, suspensions, dispersions and emulsions; injectable formulations, such as solutions, dispersions and lyophilized powders. The formulation may be suitable for immediate-release, sustained-release or controlled-release of the active ingredient. The formulation may be a regular, dispersible, chewable, orally soluble or rapidly dissolving form.

The pharmaceutical composition may be prepared by methods commonly known to those skilled in the art. In preparation of the pharmaceutical composition, an ozanimod addition salt or a crystalline ozanimod addition salt from of the present invention is mixed with one or more pharmaceutically acceptable excipients, optionally with other pharmaceutically acceptable ozanimod polymorphs, amorphous ozanimod, and ozanimod salts, optionally with one or more other active ingredients. Solid formulations may be prepared by direct mixing, granulation and other processes.

Furthermore, the third technical problem solved by the present invention is to provide one or more addition salts of ozanimod or their crystalline forms prepared by using preparation methods of the present invention for treating and/or preventing one or more diseases or adverse conditions. The diseases or adverse conditions are associated with modulation, activation, stimulation, inhibition or antagonization of selective sphingosine-1-phosphate receptor.

The diseases or adverse conditions include multiple sclerosis, transplant rejection or acute respiratory distress syndrome. The diseases or adverse conditions include multiple sclerosis, ulcerative colitis, arthritis, transplant rejection or acute respiratory distress syndrome.

Specifically, the diseases or adverse conditions include but not limited to, rejection of transplanted organs or tissues; graft-versus-host disease caused by transplantation; autoimmune syndrome, including rheumatoid arthritis; acute respiratory distress syndrome; adult respiratory distress syndrome; influenza; cancers such as lung cancer, lymphoma and blood cancer; systemic lupus erythematosus; Hashimoto thyroiditis; lymphocytic thyroiditis; multiple sclerosis; myasthenia gravis; type I and type II diabetes; meningitis; encephalomyelitis; chronic graft vascular disease; autoimmune disease after infection, including rheumatic fever and glomerulonephritis after infection; osteomyelitis; skin diseases associated with immune abnormalities such as dermatitis, warts, urticaria, acne, alopecia areata, skin cancer, etc.; eye diseases such as conjunctivitis, keratitis, scleritis, cataracts, etc.; sarcoidosis; allergies; lung and respiratory diseases such as asthma, bronchitis, emphysema, etc.; gastrointestinal diseases such as gastric ulcer, enteritis, Crohn's disease , ulcerative colitis, etc.; cardiovascular diseases such as vascular injury, arteriosclerosis, myocarditis, myocardial infarction, and vascular embolism, etc.; migraine; rhinitis; kidney nephropathy, nephritis and renal insufficiency; neuritis; polyneuritis; hyperthyroidism; leukemia; anemia; osteoporosis; necrotizing granuloma; obesity; eosinophilic fasciitis; disease; hepatitis, cirrhosis and abnormal liver function.

In further embodiments, the diseases or conditions are one or more of the following: rejection of transplanted organs or tissues; graft-versus-host disease caused by transplantation; autoimmune syndrome including arthritis, multiple sclerosis, myasthenia gravis; pollen allergy; diabetes; psoriasis; Crohn's disease; ulcerative colitis; acute respiratory distress syndrome; adult respiratory distress syndrome; influenza; autoimmune disease after infection, including rheumatic fever and glomerulonephritis after infection; cancer and cancer metastasis.

In further embodiments, the diseases or conditions are one of the following diseases:

multiple sclerosis, ulcerative colitis, arthritis, transplant rejection or acute respiratory distress syndrome. The ozanimod addition salts and their crystalline forms mainly include ozanimod besylate salt and its crystallin Form 1, ozanimod citrate salt and its crystalline Form 1, ozanimod hemi-L-malate salt and its crystalline Form 1, ozanimod dihydrogen phosphate salt and its crystalline Form 1, ozanimod hydrosulfate salt and its crystalline Form 1, ozanimod hemi-sulfate salt and its crystalline Form 1, ozanimod L-tartrate salt and its crystalline Form 1, ozanimod hemi-fumarate salt and its crystalline Form 1, ozanimod fumarate salt and its crystalline Form 1, ozanimod maleate salt and its crystalline Form 1, ozanimod hydrobromide salt and its crystalline Form 1 and ozanimod mesylate salt and its crystalline Form 1.

Furthermore, the present invention provides a treating and/or preventing method for one or more diseases or adverse conditions, which comprises administrating patients in need thereof a therapeutically and/or effectively amount of an ozanimod salt or a crystalline ozanimod salt form, or pharmaceutical composition thereof in the present invention. The diseases or adverse conditions are associated with modulating, activation, stimulation, inhibition or antagonizing of selective sphingosine-1-phosphate (SIP) receptor. The diseases or adverse conditions include multiple sclerosis, ulcerative colitis, arthritis, transplant rejection or acute respiratory distress syndrome and so on. The patients include but not limit to mammals.

### DESCRIPTION OF DRAWING

FIG. 1 is an X-ray powder diffraction pattern of the known ozanimod hydrochloride prepared by referencing the methods described in example [0397] of patent CN102762100B.

FIG. 2 is an X-ray powder diffraction pattern of ozanimod besylate Form 1 of the present invention.

FIG. 3 is a DSC thermogram of ozanimod besylate Form 1 of the present invention.

FIG. 4 is a TGA thermogram of ozanimod besylate Form 1 of the present invention.

FIG. 5 is an isothermal adsorption curve of ozanimod besylate Form 1 of the present invention.

FIG. 6 is a PLM plot of ozanimod besylate Form 1 of the present invention.

FIG. 7 is an IR spectrum of ozanimod besylate Form 1 of the present invention.

FIG. 8 is an X-ray powder diffraction pattern of ozanimod citrate Form 1 of the present invention.

FIG. 9 is a DSC thermogram of ozanimod citrate Form 1 of the present invention.

FIG. 10 is a TGA thermogram of ozanimod citrate Form 1 of the present invention.

FIG. 11 is an isothermal adsorption curve of ozanimod citrate Form 1 of the present invention.

FIG. 12 is a PLM plot of ozanimod citrate Form 1 of the present invention.

FIG. 13 is an IR spectrum of ozanimod citrate Form 1 of the present invention.

FIG. 14 is an X-ray powder diffraction pattern of ozanimod hemi-L-malate salt Form 1 of the present invention.

FIG. 15 is a DSC thermogram of ozanimod hemi-L-malate Form 1 of the present invention.

FIG. 16 is a TGA thermogram of ozanimod hemi-L-malate Form 1 of the present invention.

FIG. 17 is an isothermal adsorption curve of ozanimod hemi-L-malate Form 1 of the present invention.

FIG. 18 is a PLM plot of ozanimod hemi-L-malate Form 1 of the present invention.

FIG. 19 is an IR spectrum of ozanimod hemi-L-malate Form 1 of the present invention.

FIG. 20 is an X-ray powder diffraction pattern of ozanimod dihydrogen phosphate Form 1 of the present invention.

FIG. 21 is a DSC thermogram of ozanimod dihydrogen phosphate Form 1 of the present invention.

FIG. 22 is a TGA thermogram of ozanimod dihydrogen phosphate Form 1 of the present invention.

FIG. 23 is an isothermal adsorption curve of ozanimod dihydrogen phosphate Form 1 of the present invention.

FIG. 24 is a PLM plot of ozanimod dihydrogen phosphate Form 1 of the present invention.

FIG. 25 is an IR spectrum of ozanimod dihydrogen phosphate Form 1 of the present invention.

FIG. 26 is an X-ray powder diffraction pattern of ozanimod hydrosulfate Form 1 of the present invention.

FIG. 27 is a DSC thermogram of ozanimod hydrosulfate Form 1 of the present invention.

FIG. 28 is a TGA thermogram of ozanimod hydrosulfate Form 1 of the present invention.

FIG. 29 is an isothermal adsorption curve of ozanimod hydrosulfate Form 1 of the present invention.

FIG. 30 is a PLM plot of ozanimod hydrosulfate Form 1 of the present invention.

FIG. 31 is an IR spectrum of ozanimod hydrosulfate Form 1 of the present invention.

FIG. 32 is an X-ray powder diffraction pattern of ozanimod hemi-sulfate Form 1 of the present invention.

FIG. 33 is a DSC thermoram of ozanimod hemi-sulfate Form 1 of the present invention.

FIG. 34 is a TGA thermogram of ozanimod hemi-sulfate Form 1 of the present invention.

FIG. 35 is an isothermal adsorption curve of ozanimod hemi-sulfate Form 1 of the present invention.

FIG. 36 is a PLM plot of ozanimod hemi-sulfate Form 1 of the present invention.

FIG. 37 is an IR spectrum of ozanimod hemi-sulfate Form 1 of the present invention.

FIG. 38 is an X-ray powder diffraction pattern of ozanimod L-tartrate Form 1 of the present invention.

FIG. 39 is a DSC thermogram of ozanimod L-tartrate Form 1 of the present invention.

FIG. 40 is a TGA thermogram of ozanimod L-tartrate Form 1 of the present invention.

FIG. 41 is an isothermal adsorption curve of ozanimod L-tartrate Form 1 of the present invention.

FIG. 42 is a PLM plot of ozanimod L-tartrate Form 1 of the present invention.

FIG. 43 is an IR spectrum of ozanimod L-tartrate Form 1 of the present invention.

FIG. 44 is an X-ray powder diffraction pattern of ozanimod hemi-fumarate Form 1 of the present invention.

FIG. 45 is a DSC thermogram of ozanimod hemi-fumarate Form 1 of the present invention.

FIG. 46 is a TGA thermogram of ozanimod hemi-fumarate Form 1 of the present invention.

FIG. 47 is an isothermal adsorption curve of ozanimod hemi-fumarate Form 1 of the present invention.

FIG. 48 is a PLM plot of ozanimod hemi-fumarate Form 1 of the present invention.

FIG. 49 is an IR spectrum of ozanimod hemi-fumarate Form 1 of the present invention.

FIG. 50 is an X-ray powder diffraction pattern of ozanimod fumarate Form 1 of the present invention.

FIG. 51 is a DSC thermogram of ozanimod fumarate Form 1 of the present invention.

FIG. 52 is a TGA thermogram of ozanimod fumarate Form 1 of the present invention.

FIG. 53 is an isothermal adsorption curve of ozanimod fumarate Form 1 of the present invention.

FIG. 54 is a PLM plot of ozanimod fumarate Form 1 of the present invention.

FIG. 55 is an IR spectrum of ozanimod fumarate Form 1 of the present invention.

FIG. 56 is an X-ray powder diffraction pattern of ozanimod maleate Form 1 of the present invention.

FIG. 57 is a DSC thermogram of ozanimod maleate Form 1 of the present invention.

FIG. 58 is a TGA thermogram of ozanimod maleate Form 1 of the present invention.

FIG. 59 is an isothermal adsorption curve of ozanimod maleate salt Form 1 of the present invention.

FIG. 60 is a PLM plot of ozanimod maleate Form 1 of the present invention.

FIG. 61 is an IR spectrum of ozanimod maleate Form 1 of the present invention.

FIG. 62 is an X-ray powder diffraction pattern of ozanimod hydrobromide Form 1 of the present invention.

FIG. 63 is a DSC thermogram of ozanimod hydrobromide Form 1 of the present invention.

FIG. 64 is a TGA thermogram of ozanimod hydrobromide Form 1 of the present invention.

FIG. 65 is an isothermal adsorption curve of ozanimod hydrobromide Form 1 of the present invention.

FIG. 66 is a PLM plot of ozanimod hydrobromide Form 1 of the present invention.

FIG. 67 is an IR spectrum of ozanimod hydrobromide Form 1 of the present invention.

FIG. 68 is an X-ray powder diffraction pattern of ozanimod mesylate Form 1 of the present invention.

FIG. 69 is a DSC thermogram of ozanimod mesylate Form 1 of the present invention.

FIG. 70 is a TGA thermogram of ozanimod mesylate Form 1 of the present invention.

FIG. 71 is an isothermal adsorption curve of ozanimod mesylate Form 1 of the present invention.

FIG. 72 is a PLM plot of ozanimod mesylate Form 1 of the present invention.

FIG. 73 is an IR spectrum of ozanimod mesylate Form 1 of the present invention.

FIG. 74 is an isothermal adsorption curve of the known ozanimod hydrochloride prepared by referencing the methods described in example [0397] of patent CN102762100B.

### SPECIFIC IMPLEMENTATIONS

The following examples help to further understand the present invention, but are not intended to limit the contents of the present invention.

Instruments and characterization methods:

X-ray powder diffraction (XRPD): performed on Bruker D8 Advance diffractometer. Samples were tested at room temperature. Testing conditions: 2θ scan range 3-40°, step size 0.02°, and speed 0.2 s/step.

Differential thermal analysis data were collected on TA Instruments Q200 DSC. The testing conditions: place 1 mg to 10 mg sample into an aluminum pan with a pinholed lid, ramp to 200° C.-250° C. at a rate of 10° C./min under the protection of dry N2 at a purge rate of 40 mL/min.

Thermogravimetric analysis data were collected on TA Instruments Q500 TGA. The testing conditions: place 5 mg to 15 mg sample into a platinum pan, use high resolution method, ramp to 350° C. at a rate of 10° C./min under the protection of dry N₂ at a purge rate of 40 mL/min.

Dynamic vapor sorption data and isothermal sorption data are collected on TA Instruments Q5000 TGA. A sample of 1 to 10 mg is usually placed in a platinum pan; and the TA software records the change in weight of the sample during relative humidity from 0% to 80% to 0%. Depending on the specifics of the sample, different adsorption and desorption steps are also applied to the sample.

The Infrared spectrometry (IR) data are collected on Bruker Tensor 27 equipped with an attenuated total reflection (ATR). The infrared spectra are collected over the range of 600 cm⁻¹ to 4000 cm⁻¹.

¹H Nuclear magnetic resonance spectrum (1H-NMR) data are collected on Bruker Avance II DMX 300 MHz. Place 1 mg to 5 mg sample into NMR tube, dissolve with 0.5 mL deuterium reagents.

IC data was taken from the Dionex ICS-900 ion chromatograph.

The examples were operated at room temperature unless otherwise noted, and the solvent ratios were all volume ratios.

All reagents used in the examples were commercially purchased.

### Preparation Example 1

Ozanimod was prepared according to the methods described in examples [0388-0399] of the patent document CN102762100B.

¹HNMR (300 MHz, CDC13—CH3OD) 8.33(d,1H),8.26(dd,1H),7.99(d,1H),7.47(d,1H), 7.33(t,1H),7.07(d,1H),4.74(t,1H),4.27(t,1H),3.62 (q,2H),3.37-3.32 (m, 1H),3.18-3.10 (m, 1H), 2.79 (t,2H), 2.48-2.37 (m, 1H),1.92-1.85 (m, 1H), 1.40 (d, 6H), indicating that it was the known ozanimod.

### Preparation Example 2

Ozanimod hydrochloride was prepared according to the method described in example

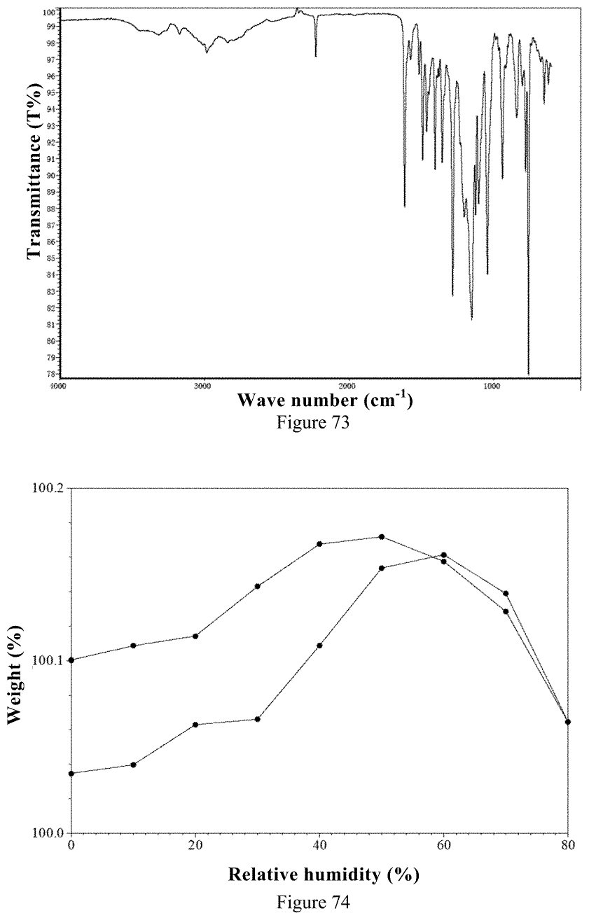

of the patent document CN102762100B. ¹HNMR (500MHz, DMSO-d6): δ9.19(s,2H),8.54 (s,1H),8.42(d, J=9.1Hz,1H), 8.17(d, J=7.7Hz,1H),7.96(d, J=7.5Hz,1H), 7.58(t, J=7.4Hz,2H),5.27(t, J=4.7Hz,1H), 4.99(dt, J=11.8,6.0Hz,1H), 4.92(s,1H), 3.72(q, J=4.3Hz,2H), 3.50(dt, J=16.1,7.5Hz,1H), 3.04(d,J=27.0Hz,2H), 2.31(ddd, J=12.9,7.8,3.6Hz,1H), 1.39(d, J=5.8Hz,6H). It indicated that it was the known ozanimod hydrochloride. The hydrochloride was crystalline. Its X-ray powder diffraction pattern is shown in FIG. 1, and its isotherm adsorption curve is shown in FIG. 74.

### Example 1

Took 2 g ozanimod free base of Preparation Example 1, added 300 mL of n-propanol, applied ultrasonical force to dissolve it; weighed 938 mg of benzenesulfonic acid, added 40 mL of n-propanol, applied ultrasonical force to dissolve it; and then the acid solution was added dropwise to the free base, stirred at room temperature to precipitate crystals, after 3 days, filtrated, and then vacuum dried at room temperature for 24 hours to obtain 2.61 g ozanimod besylate Form 1; 74% yield.

### Example 2

Took 20 mg ozanimod free base of Preparation Example 1, added 0.3 mL of acetone, applied ultrasonical force to dissolve it; weighed 7.82 mg of benzenesulfonic acid, added 0.3 mL of acetone, applied ultrasonical force to dissolve it; and then the acid solution was added dropwise to the free base, stirred at room temperature to precipitate crystals, after 1 day, filtrated, and then vacuum dried at room temperature for 16 hours to obtain 25.6 mg ozanimod besylate Form 1; 92% yield.

### Example 3

Took 20 mg ozanimod free base of preparation example 1, added 1.5 mL of a mixed solvent of n-propanol and acetone (1:1 v/v), applied ultrasonical force to dissolve it; weighed 11.73 mg of benzenesulfonic acid, added 0.2 mL of a mixed solvent of n-propanol and acetone (1:1 v/v), applied ultrasonical force to dissolve it; and then the acid solution was added dropwise to the free base, stirred at 40° C. to precipitate crystals, after 7 days, filtrated, and then vacuum dried at room temperature for 48 hours to obtain 23.3 mg ozanimod besylate Form 1; 83% yield.

### Example 4

Took 20 mg ozanimod free base of Preparation Example 1, added 1 mL of ethanol, applied ultrasonical force to dissolve it; weighed 10.17 mg of benzenesulfonic acid, added 0.2 mL of ethanol, applied ultrasonical force to dissolve it; and then the acid solution was added dropwise to the free base, stirred at room temperature to precipitate crystals, after 5 days, filtrated, and then vacuum dried at 10° C. for 16 hours to obtain 23.8 mg ozanimod besylate Form 1; 83% yield.

### Example 5

Ozanimod besylate Form 1 can also be obtained by replacing solvents, stirring temperature and stirring time in Examples 1 to 4 with the following table.

| | | Solvent 2 | Stirring | | |
|---|---|---|---|---|---|
| | | (benzene- | temper- | Stirring | |
| Experiment | Solvent 1 | sulfonic | ature | time | Yield |
| No. | (ozanimod) | acid) | (° C.) | (d) | (%) |
| Experiment 1 | 2-butanol | 2-butanol | RT | 3 | 81 |
| Experiment 2 | acetone | methanol | 30 | 4 | 85 |
| Experiment 3 | butanone | acetone | 15 | 2 | 86 |
| Experiment 4 | n-butanol | butanone | 35 | 1 | 82 |
| Experiment 5 | isopropanol | acetone | 10 | 5 | 88 |
| Experiment 6 | n-propanol | ethanol | 25 | 7 | 79 |
| Experiment 7 | acetone | isopropanol | 10 | 3 | 85 |

### Example 6

Took 2 g ozanimod free base of Preparation Example 1, added 600 mL of n-propanol, applied ultrasonical force to dissolve it; weighed 1.185 g of benzenesulfonic acid, added 40 mL of n-propanol, applied ultrasonical force to dissolve it; the acid solution was then added dropwise to the free base, added 5 mL of methyl tert-butyl ether, stirred at room temperature to precipitate crystals, after 3 days, filtrated, and then vacuum dried at room temperature for 36 hours to obtain 2.54 g ozanimod besylate Form 1; 91% yield.

### Example 7

Took 2 g ozanimod free base of Preparation Example 1, added 3.0 mL of n-propanol, applied ultrasonical force to dissolve it; weighed 11.62 mg of benzenesulfonic acid, added 0.4 mL of n-propanol, applied ultrasonical force to dissolve it; the acid solution was then added dropwise to the free base, added 5 mL of methyl tert-butyl ether, stirred at room temperature to precipitate crystals, after 3 days, filtrated, and then vacuum dried at room temperature for 36 hours to obtain 22.2 mg ozanimod besylate Form 1; 79% yield.

### Example 8

Took 20 mg ozanimod free base of Preparation Example 1, added 0.3 mL of acetone, applied ultrasonical force to dissolve it; weighed 7.82 mg of benzenesulfonic acid, added 0.3 mL of acetone, applied ultrasonical force to dissolve it; the acid solution was then added dropwise to the free base, stirred at room temperature to precipitate crystals, after 1 day, filtrated, and then vacuum dried at room temperature for 16 hours to obtain 25.2 mg ozanimod besylate Form 1; 90% yield.

### Example 9

Took 20 mg ozanimod free base of Preparation Example 1, added 8.0 mL of a mixed solvent of n-propanol and acetone (1:1 v/v), applied ultrasonical force to dissolve it; weighed 9.39 mg of benzenesulfonic acid, added 0.6 mL of a mixed solvent of n-propanol and acetone (1:1 v/v) , applied ultrasonical force to dissolve it; the acid solution was then added dropwise to the free base, added 3 mL of ethyl acetate , stirred at 40° C. to precipitate crystals , after 1 day, filtrated, and then vacuum dried at room temperature for 48 hours to obtain 23.6mg ozanimod besylate slat form 1; 84% yield.

### Example 10

Ozanimod besylate Form 1 can also be obtained by replacing solvents, stirring temperature and stirring time in Examples 6 to 9 with the following table.

| | | Co-solvent 2 | | Stirring | Stirring | |
|---|---|---|---|---|---|---|
| Experiment | Co-solvent 1 | (benzenesulfonic | Solvent 3 | temperature | time | Yield |
| No. | (ozanimod) | acid) | (antisolvent) | (° C.) | (day) | (%) |
| Experiment 1 | butanone | ethanol | ethyl | 20 | 2 | 80 |
| | | | acetate | | | |
| Experiment 2 | methanol/ | methanol | isopropyl | 30 | 4 | 77 |
| | acetone | | ether | | | |
| Experiment 3 | 2-butanol | acetone | isopropyl | 15 | 3 | 76 |
| | | | acetate | | | |
| Experiment 4 | butanone | butanone | hexane | 35 | 1 | 67 |
| Experiment 5 | isopropanol | acetone | MTBE | 10 | 5 | 87 |
| Experiment 6 | n-propanol | ethanol | n-heptane | RT. | 7 | 83 |

The ¹H-NMR data, XRPD patterns, DSC thermograms, TGA thermograms, IR spectra of the samples prepared in Examples 2 to 10 are the same as or similar to that of the sample prepared in Example 1, indicating the salt and the crystalline form obtained in Examples 2 to 10 are the same as Example 1.

### Examples 11-90

Referring to Examples 1 to 10, the benzenesulfonic acid was sequentially replaced with the same mole of citric acid, phosphoric acid, sulfuric acid, L-tartaric acid, fumaric acid, maleic acid, hydrobromic acid and methanesulfonic acid to obtain ozanimod citrate Form 1, ozanimod dihydrogen phosphate Form 1, ozanimod hydrosulfate Form 1, ozanimod L-tartrate Form 1, ozanimod fumarate Form 1, ozanimod maleate Form 1, ozanimod hydrobromide Form 1 and ozanimod mesylate Form 1, respectively.

### Example 91

The stoichiometry of the crystalline ozanimod addition salts in Examples 1 to 90 is confirmed by 1HNMR or ion chromatography, and some results are shown in the following table.

| | | | Salt molar |
|---|---|---|---|
| | | Characterization Data | (base:acid) |
| Besylate | ¹H-NMR | ¹HNMR(500 MHz, DMSO-d6): δ8.90(s, 2H), | 1:1 |
| | | 8.52(d, J = 1.9 Hz, 1H), 8.41(dd, J = 8.9, 1.9 Hz, 1 | |
| | | H), 8.17(d, J = 7.7 Hz, 1H), 7.88(d, J = 7.6 Hz, 1 | |
| | | H), 7.65-7.52(m, 4H), 7.31(d, J = 6.8 Hz, 3H), | |
| | | 5.26(s, 1H), 5.04-4.87(m, 2H), 3.75-3.64(m, 2 | |
| | | H), 3.48(dt, J = 15.1, 7.9 Hz, 1H), 3.32-3.21(m, 2 | |
| | | H), 3.13-2.97(m, 2H), 2.29(dt, J = 9.5, 4.4 Hz, 1 | |
| | | H), 1.39(d, J = 6.0 Hz, 6H) | |
| Citrate | ¹H-NMR | ¹HNMR(500 MHz, DMSO-d6): δ8.53-8.45 | 1:1 |
| | | (m, 1H), 8.44-8.35(m, 1H), 8.07(d, J = 7.7 Hz, 1 | |
| | | H), 7.74(d, J = 7.5 Hz, 1H), 7.60-7.44(m, 2H), | |
| | | 4.98(p, J = 6.1 Hz, 1H), 4.59(t, J = 6.3 Hz, 1H), 3.61 | |
| | | (t, J = 5.5 Hz, 2H), 3.41(dt, J = 13.5, 6.8 Hz, 2H), | |
| | | 3.17(dt, J = 16.9, 7.6 Hz, 1H), 2.88(hept, J = 6.8, 5.8 | |
| | | Hz, 2H), 2.55(d, J = 15.0 Hz, 1H), 2.08(dt, J = | |
| | | 14.0, 6.8 Hz, 1H), 1.39(d, J = 6.0 Hz, 6H) | |
| Dihydrogen | Ion chromatography | In present invention, the phosphate ion | 1:1 |
| phosphate | | content of ozanimod dihydrogen phosphate | |
| | | is 18.7%. Theoretical phosphate ion content | |
| | | of ozanimod dihydrogen phosphate is | |
| | | 19.3%. | |
| | | The theoretical phosphate ion content of | |
| | | ozanimod hydrogen phosphate is 10.5%. | |
| Hydrosulfate | Ion chromatography | In present invention, the sulfate ion content | 1:1 |
| | | of ozanimod hydrosulfate is 10.8%. The | |
| | | theoretical sulfate ion content of ozanimod | |
| | | hemi-sulfate is 19.1%. The theoretical | |
| | | sulfate ion content of ozanimod | |
| | | hydrosulfate is 10.6%. | |
| L-tartrate | ¹H-NMR | ¹HNMR(500 MHz, DMSO-d6): δ8.50(d, J = | 1:1 |
| | | 2.2 Hz, 1H), 8.40(dd, J = 9.0, 2.2 Hz, 1H), 8.06(d, J = | |
| | | 7.6 Hz, 1H), 7.74(d, J = 7.5 Hz, 1H), 7.55(d, J = | |
| | | 9.2 Hz, 1H), 7.49(t, J = 7.6 Hz, 1H), 4.98(dt, J = | |
| | | 12.1, 6.0 Hz, 2H), 4.64-4.52(m, 2H), 3.91(s, 2H), | |
| | | 3.60(t, J = 5.5 Hz, 2H), 3.46-3.35(m, 1H), 3.16(dt, | |
| | | J = 17.1, 7.5 Hz, 1H), 2.86(tt, J = 12.2, 5.7 Hz, 2 | |
| | | H), 2.45(dd, J = 13.4, 5.3 Hz, 1H), 2.06(dq, J = | |
| | | 13.5, 5.8 Hz, 1H), 1.39(d, J = 6.0 Hz, 6H) | |
| Fumarate | ¹H-NMR | ¹HNMR(500 MHz, DMSO-d6)δ:8.51 (s, 1H), | 1:1 |
| | | 8.41(d, J = 11.1 Hz, 1H), 8.08(d, J = 7.6 Hz, 1H), | |
| | | 7.77(d, J = 7.5 Hz, 1H), 7.56(d, J = 9.2 Hz, 1H), | |
| | | 7.50(t, J = 7.6 Hz, 1H), 6.52(s, 2H), 4.98(p, J = | |
| | | 6.0 Hz, 1H), 4.67-4.57(m, 1H), 3.62(t, J = 5.5 Hz, 3H), | |
| | | 3.48-3.37(m, 2H), 3.18(dt, J = 17.1, 7.4 Hz, 2H), | |
| | | 2.88(ddq, J = 17.9, 12.0, 5.5 Hz ,2H), 2.48-2.42 | |
| | | (m, 1H), 2.10(dq, J = 13.4, 5.5 Hz, 1H), 1.39(d, J = | |
| | | 6.0 Hz, 6H) | |
| Maleate | ¹H-NMR | ¹HNMR(500 MHz, DMSO-d6)δ8.90(s, 2H), | 1:1 |
| | | 8.52(d, J = 2.2 Hz, 1H), 8.41(dd, J = 9.0, 2.2 Hz, 1 | |
| | | H), 8.17(d, J = 7.6 Hz, 1H), 7.87(d, J = 7.6 Hz, 1 | |
| | | H), 7.63-7.53(m, 2H), 6.02(s, 2H), 5.27(s, 1H), | |
| | | 5.06-4.85(m, 2H), 3.69(s, 2H), 3.48(dt, J = 15.2, | |
| | | 8.1 Hz, 1H), 3.05(ddt, J = 38.9, 12.3, 5.1 Hz, 2H), | |
| | | 2.56(dt, J = 14.8, 7.5 Hz, 1H), 2.29(ddt, J = 13.4, | |
| | | 8.7, 4.5 Hz, 1H), 1.39(d, J = 6.0 Hz, 6H) | |
| Hydrobromide | Ion chromatography | In present invention, the bromide ion | 1:1 |
| | | content of ozanimod hydrobromide is | |
| | | 15.8%. The theoretical bromide ion content | |
| | | of ozanimod hydrobromide is 16.5%. | |
| Mesylate | ¹H-NMR | ¹HNMR(500 MHz, DMSO-d6)δ8.95(s, 2H), | 1:1 |
| | | 8.52(d, J = 2.3 Hz, 1H), 8.41(dd, J = 8.9, 2.3 Hz, 1 | |
| | | H), 8.22-8.12(m, 1H), 7.89(d, J = 7.6 Hz, 1H), | |
| | | 7.66-7.50(m, 2H), 5.07-4.88(m, 2H), 3.70(t, J = | |
| | | 5.3 Hz, 2H), 3.49(ddd, J = 17.4, 8.6, 6.4 Hz, 1H), | |
| | | 3.27(ddd, J = 17.5, 9.1, 4.7 Hz, 1H), 3.15-2.97 | |
| | | (m, 2H), 2.56(ddd, J = 12.1, 8.6, 6.4 Hz, 1H), 2.32 | |
| | | (s, 3H), 1.39(d, J = 6.1 Hz, 6H) | |

### Example 92

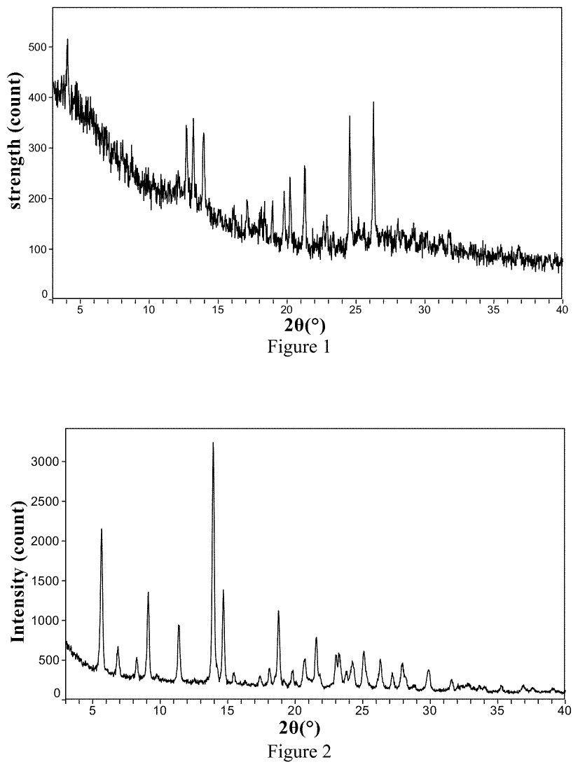

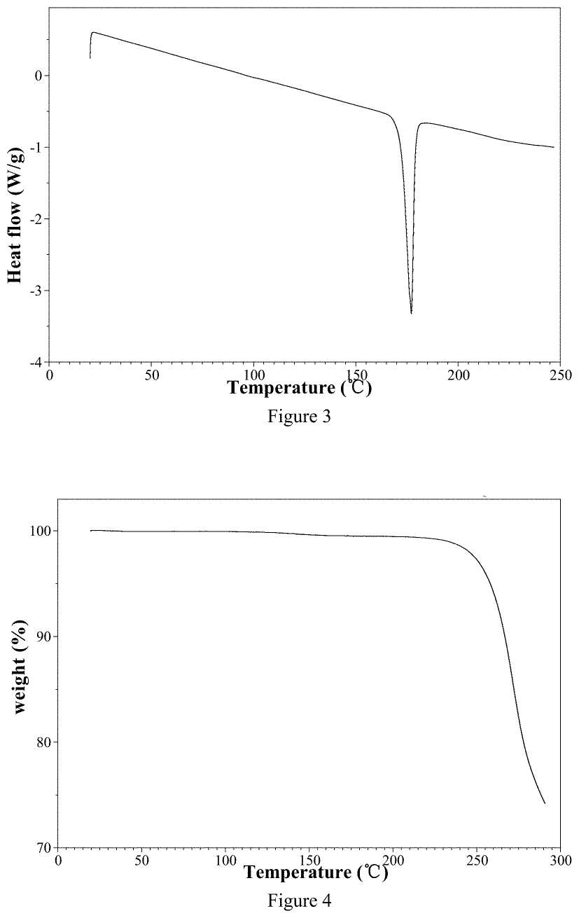

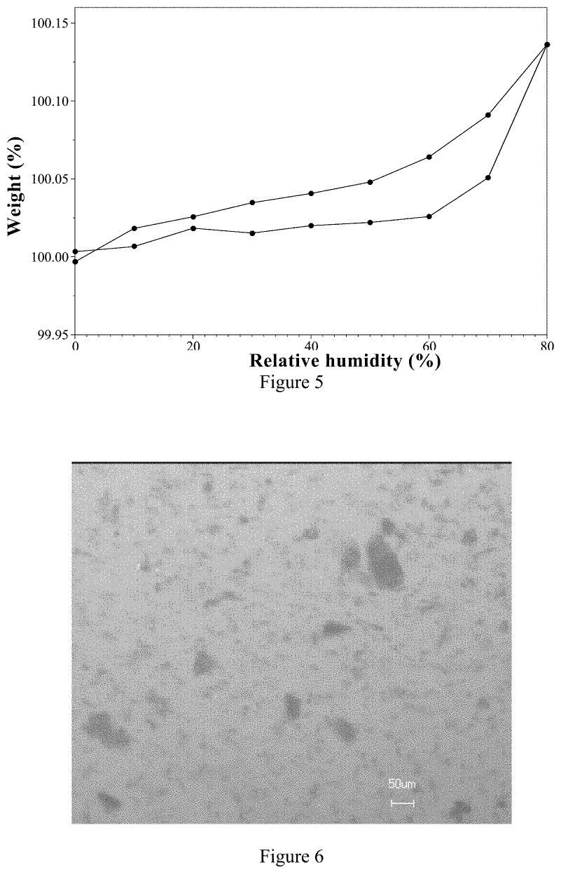

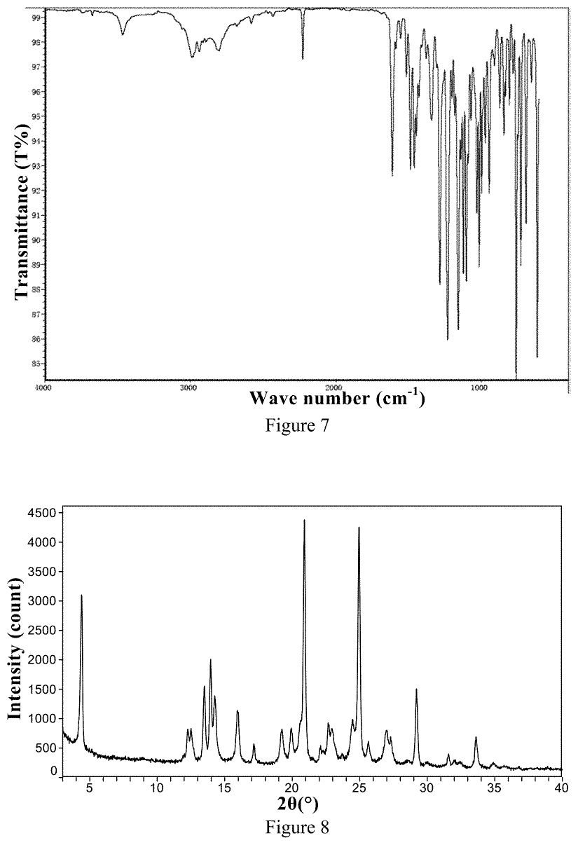

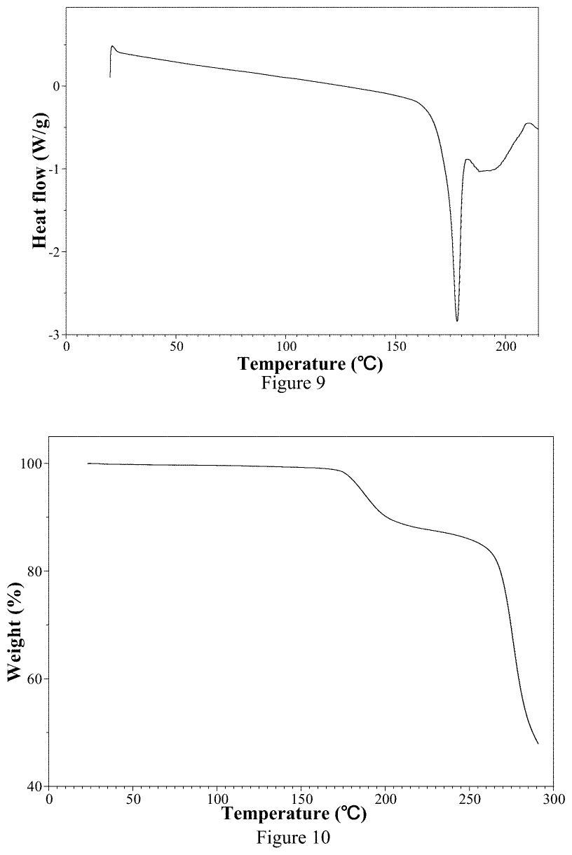

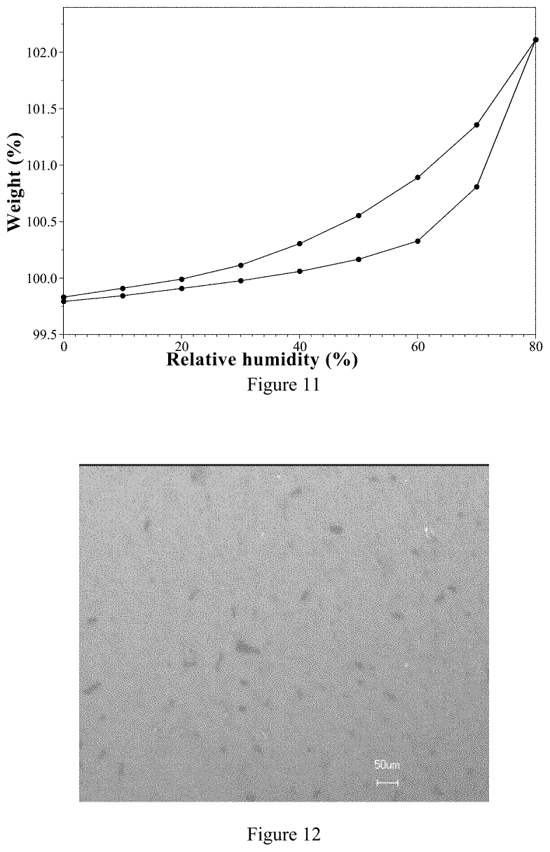

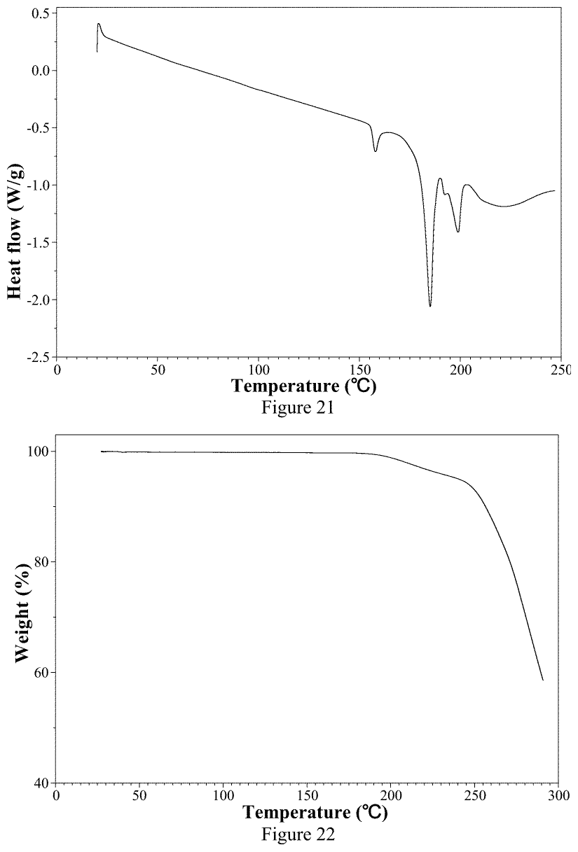

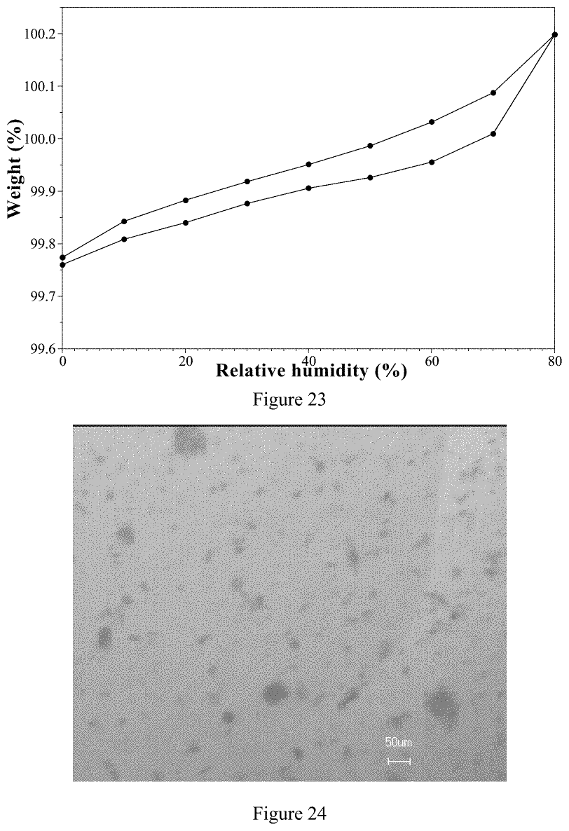

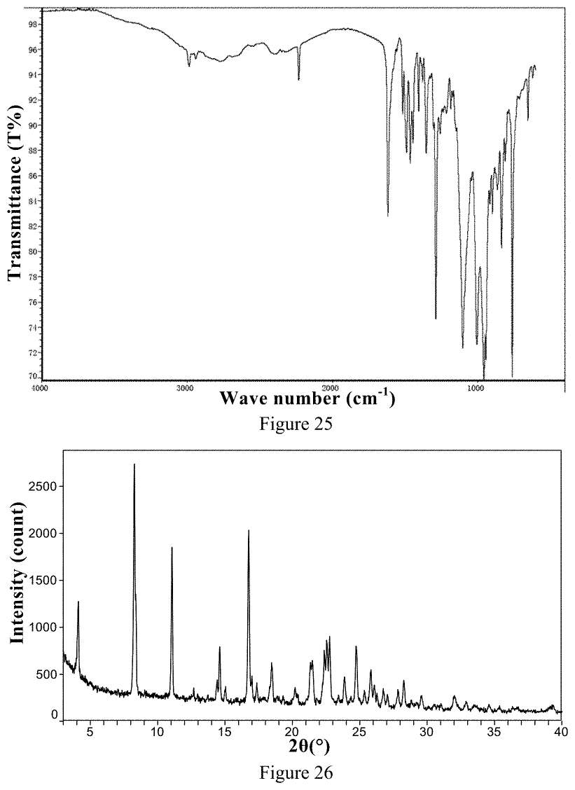

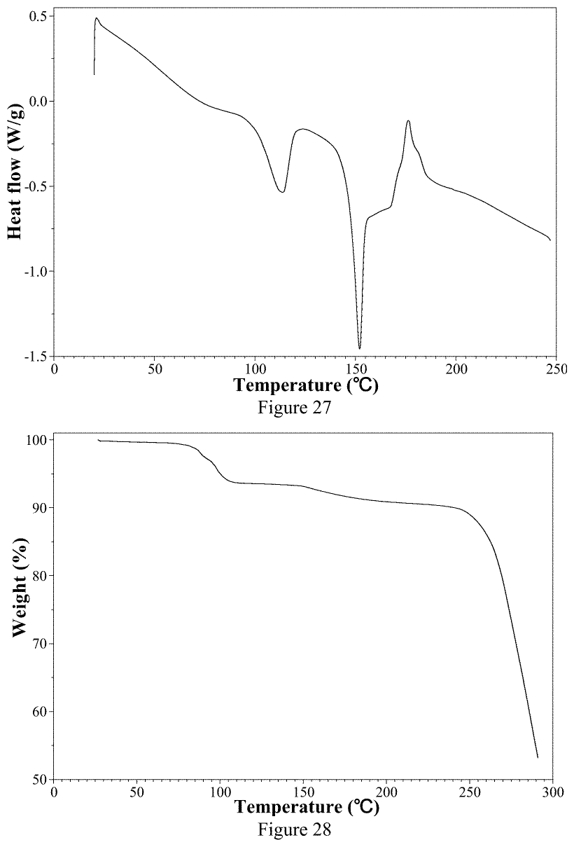

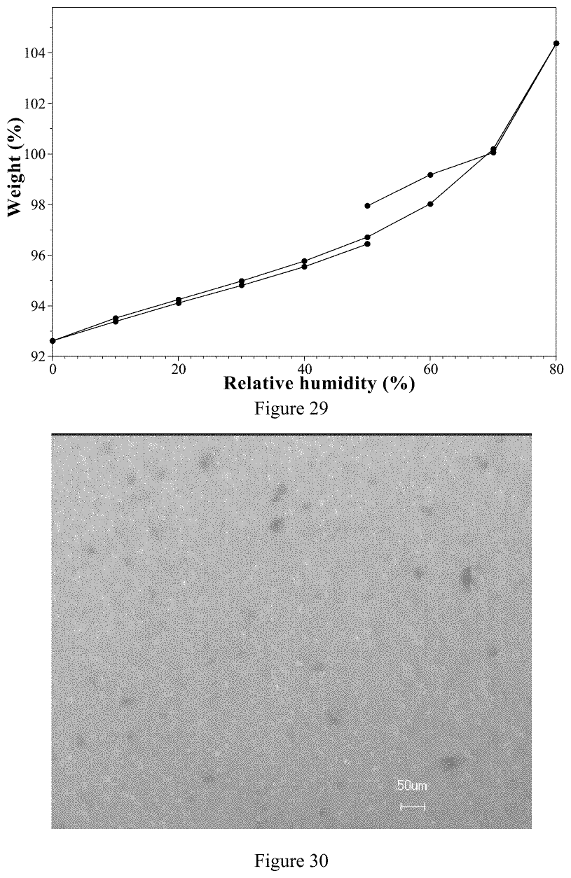

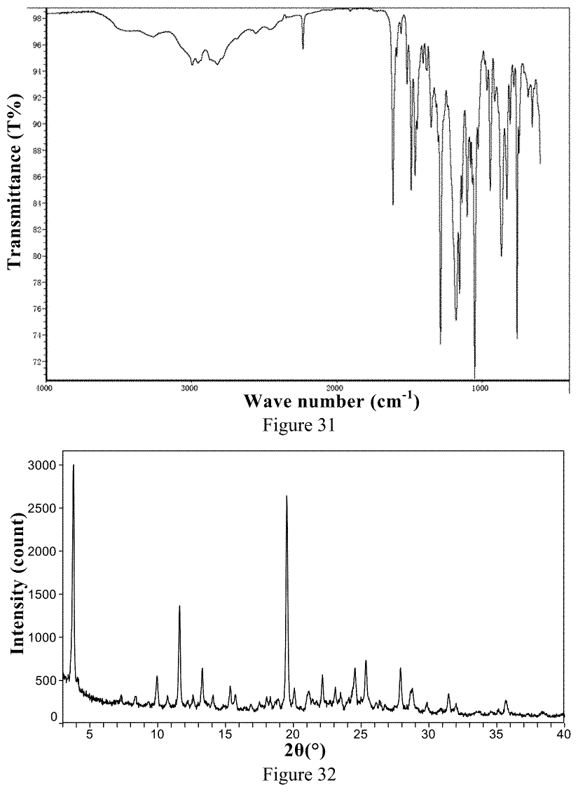

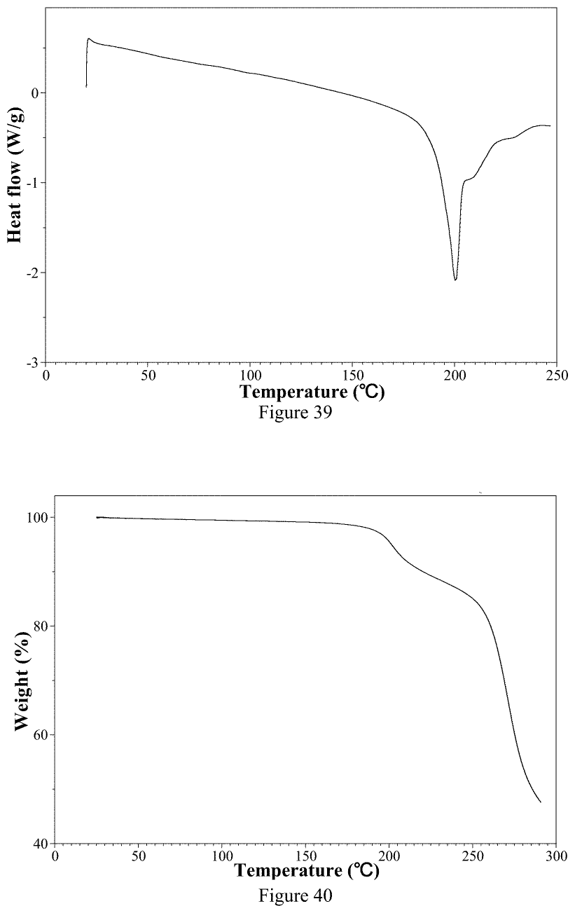

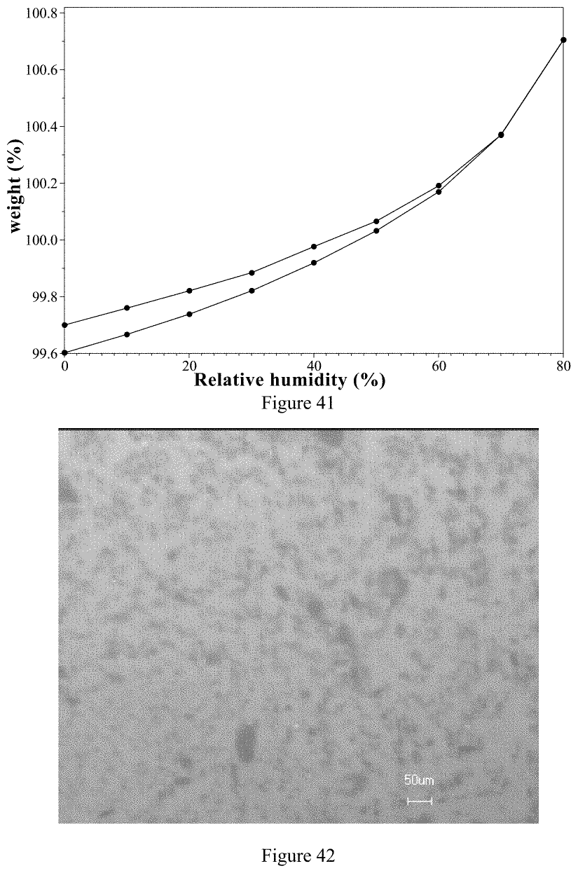

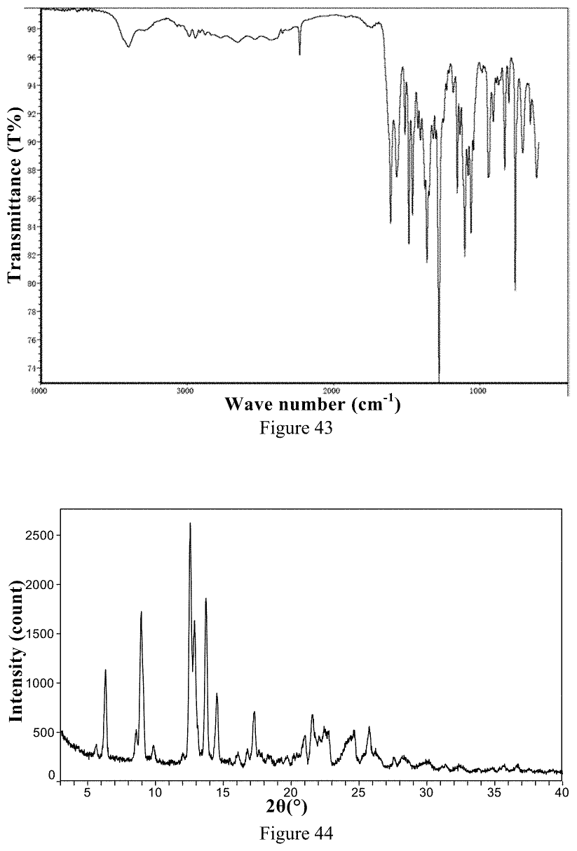

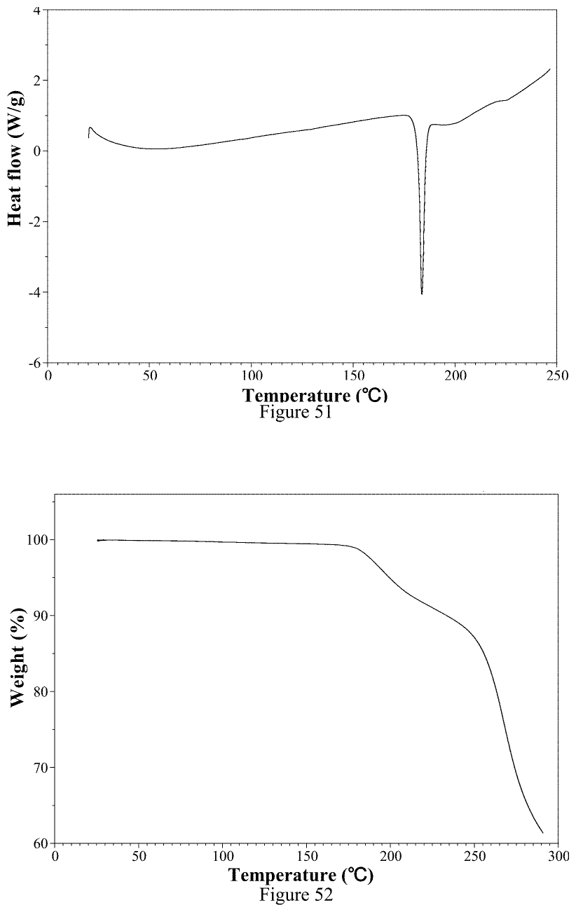

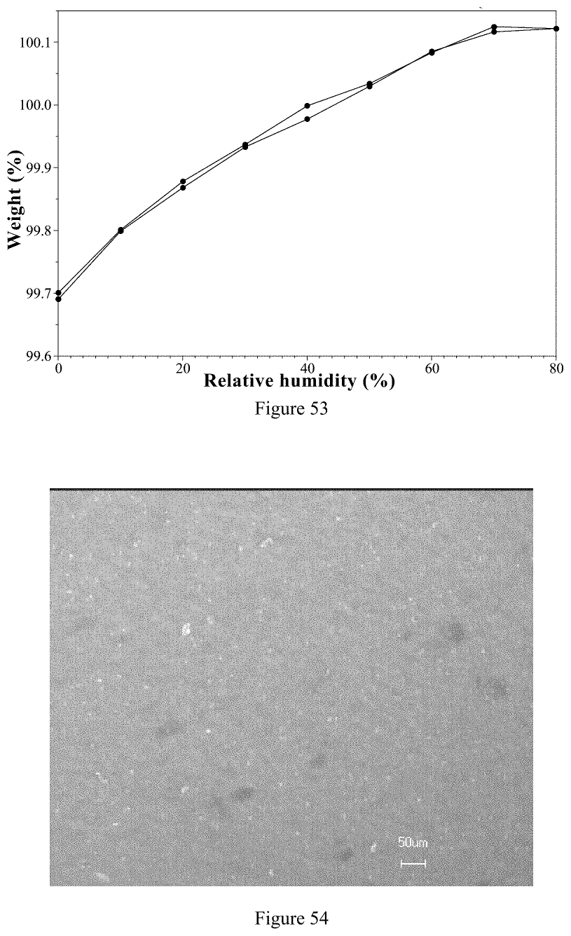

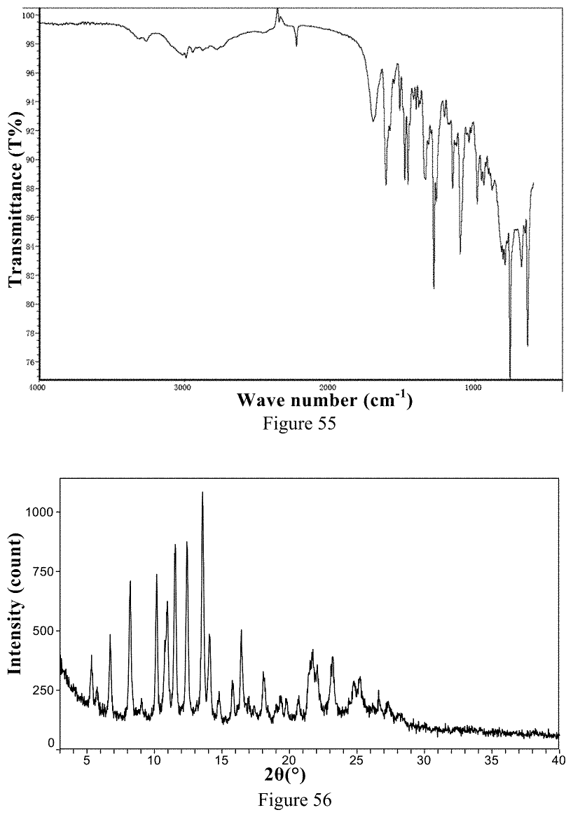

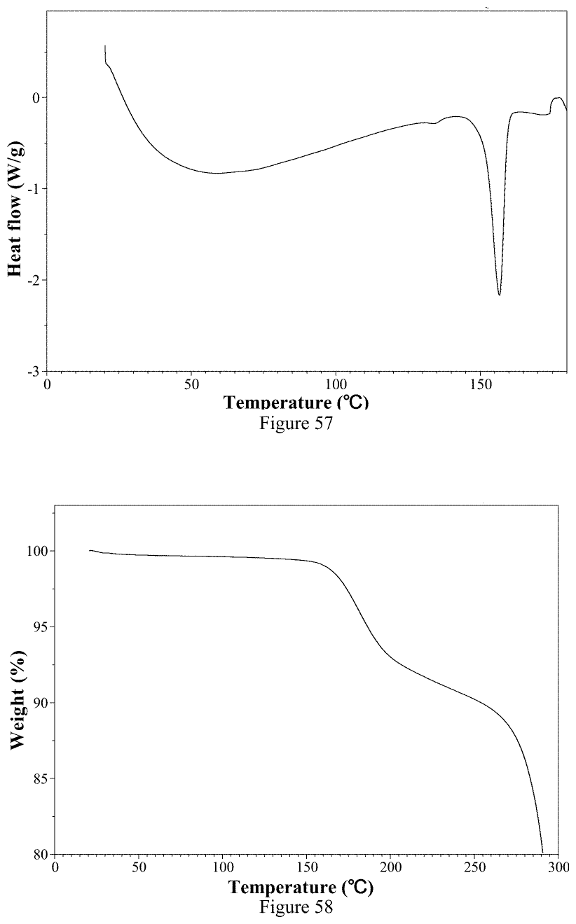

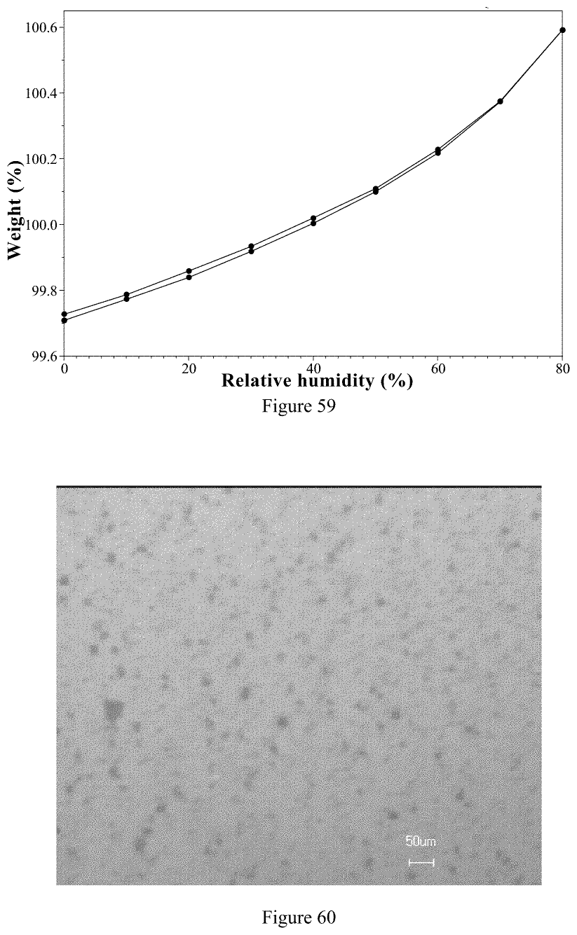

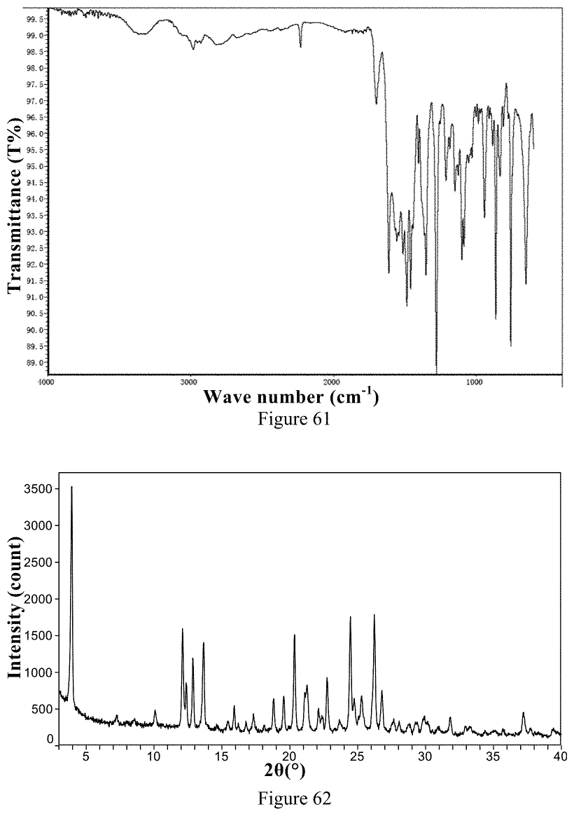

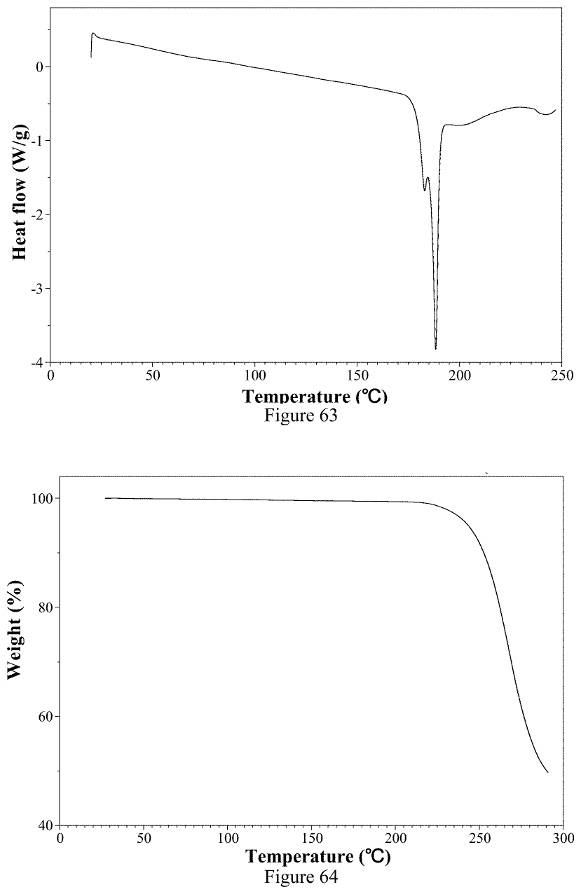

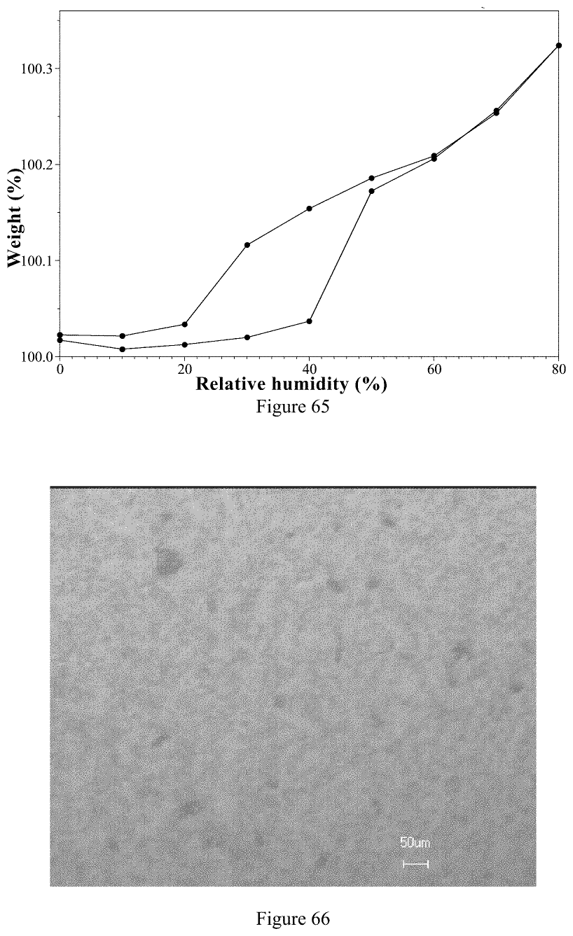

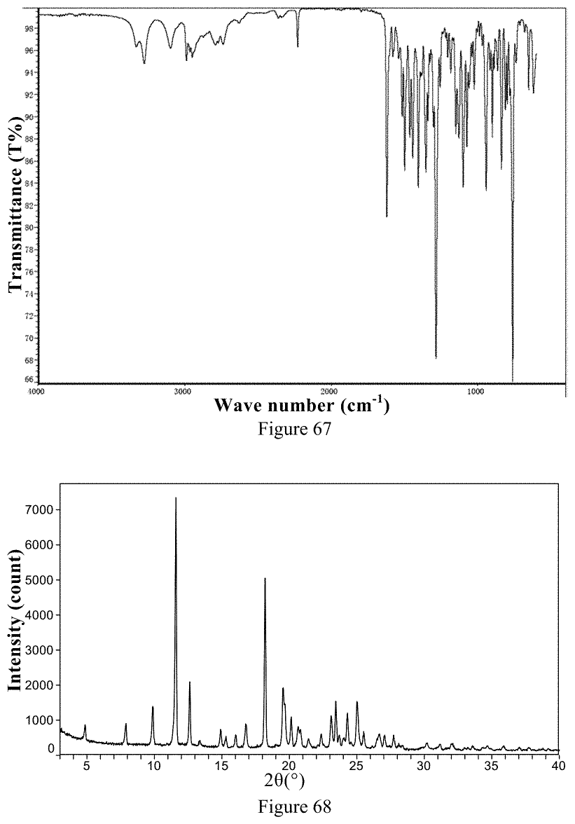

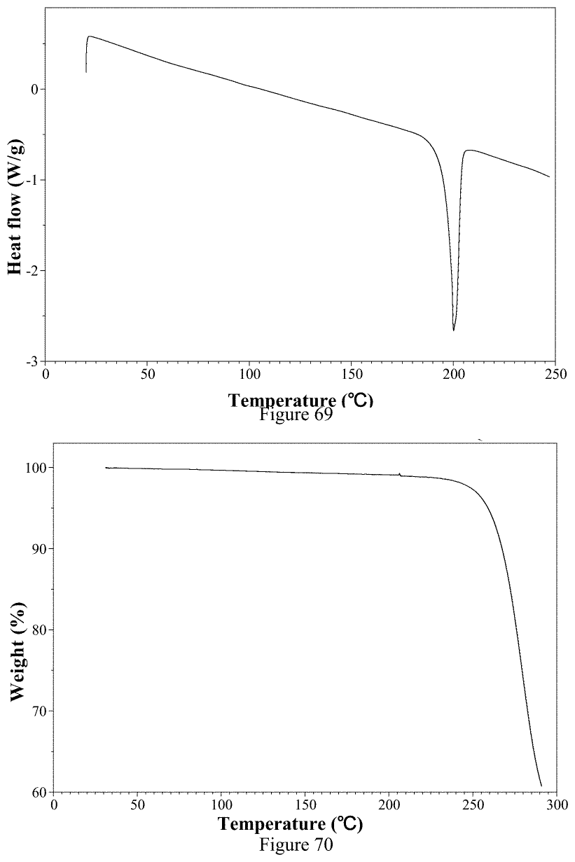

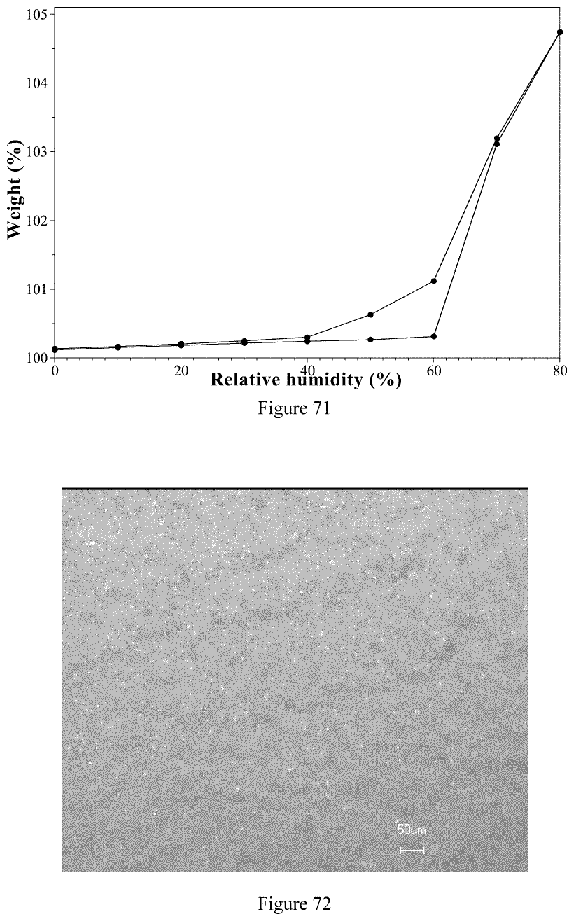

Crystalline ozanimod addition salt forms in Examples 1 to 90 were characterized by XRPD, DSC, TGA, IR and DVS, and some result are shown in the following table.

| Crystalline forms of | | | | Isothermal | | |
|---|---|---|---|---|---|---|
| ozanimod salts | XRD | DSC | TGA | adsorption | PLM | IR |
| Besylate Form 1 | FIG. 2 | FIG. 3 | FIG. 4 | FIG. 5 | FIG. 6 | FIG. 7 |
| Citrate Form 1 | FIG. 8 | FIG. 9 | FIG. 10 | FIG. 11 | FIG. 12 | FIG. 13 |
| Phosphate dihydrogen | FIG. 20 | FIG. 21 | FIG. 22 | FIG. 23 | FIG. 24 | FIG. 25 |
| Form 1 | | | | | | |
| Hydrosulfate Form 1 | FIG. 26 | FIG. 27 | FIG. 28 | FIG. 29 | FIG. 30 | FIG. 31 |
| L-tartrate Form 1 | FIG. 38 | FIG. 39 | FIG. 40 | FIG. 41 | FIG. 42 | FIG. 43 |
| Fumarate Form 1 | FIG. 50 | FIG. 51 | FIG. 52 | FIG. 53 | FIG. 54 | FIG. 55 |
| Maleate Form 1 | FIG. 56 | FIG. 57 | FIG. 58 | FIG. 59 | FIG. 60 | FIG. 61 |
| Hydrobromide Form 1 | FIG. 62 | FIG. 63 | FIG. 64 | FIG. 65 | FIG. 66 | FIG. 67 |
| Mesylate Form 1 | FIG. 68 | FIG. 69 | FIG. 70 | FIG. 71 | FIG. 72 | FIG. 73 |

### Example 93

Took 2 g ozanimod free base of Preparation Example 1, added 300 mL of n-propanol, applied ultrasonical force to dissolve it; weigh 398 mg of L-malic acid, added 40 mL of n-propanol, applied ultrasonical force to dissolve it; the acid solution was added dropwise to the free base, stirred at room temperature to precipitate crystals, after 3 days, filtrated, and then vacuum dried at room temperature for 24 hours to obtain 2.09 g ozanimod hemi-L-malate Form 1; 90% yield.

### Example 94

Took 20 mg ozanimod free base of Preparation Example 1, added 3.0 mL of acetone, applied ultrasonical force to dissolve it; weighed 3.32 mg of L-malic acid, added 0.4 mL of acetone applied ultrasonical force to dissolve it; the acid solution was added drop-wisely to the free base, stirred at room temperature to precipitate it, after 7 days, filtrated, and then vacuum dried at room temperature for 16 hours to obtain 21.1 mg ozanimod hemi-L-malate Form 1; 92% yield.

### Example 95

Took 20 mg ozanimod free base of Preparation Example 1, added 6.0 mL of n-butanol, applied ultrasonical force to dissolve it; weighed 4.64 mg of L-malic acid, added 0.6 mL of n-butanol, applied ultrasonical force to dissolve it; the acid solution was added dropwise to the free base, stirred at 10° C. to precipitate crystals, after 1 day, filtrated, and then vacuum dried at room temperature for 36 hours to obtain 19.5 mg ozanimod hemi-L-malate Form 1; 85% yield.

### Example 96

Took 20 mg ozanimod free base of Preparation Example 1, added 1.5 mL of a mixed solvent of n-propanol and acetone (1:1 v/v), applied ultrasonical force to dissolve it; weighed 5.30 mg of L-malic acid, added 0.6 mL of a mixed solvent of n-propanol and acetone (1:1 v/v), applied ultrasonical force to dissolve it; The acid solution was added dropwise to the free base, stirred at 40° C. to precipitate crystals, after 5 days, filtrated, and then vacuum dried at room temperature for 48 hours to obtain 18.8 mg ozanimod hemi-L-malate Form 1; 82% yield.

### Example 97

Ozanimod hemi-L-malate Form 1 can also be obtained by replacing solvents, stirring temperature and stirring time in Examples 93 to 96 with the following table.

| | | Solvent 2 | Stirring | Stirring | |
|---|---|---|---|---|---|
| Experiment | Solvent 1 | (L-malic | temp. | time | Yield |
| No. | (ozanimod) | acid) | (° C.) | (day) | (%) |
| Experiment 1 | ethanol | ethanol | 10 | 2 | 81 |
| Experiment 2 | 2-butanol | 2-butanol | 15 | 1 | 75 |
| Experiment 3 | acetone | methanol | 20 | 4 | 72 |
| Experiment 4 | butanone | acetone | RT | 3 | 78 |
| Experiment 5 | methanol/ | butanone | 25 | 3 | 77 |
| | butanone | | | | |
| Experiment 6 | isopropanol | acetone | 15 | 7 | 83 |

### Example 98

Took 2 g ozanimod free base of Preparation Example 1, added 600 mL of n-propanol, applied ultrasonical force to dissolve it; weighed 352 mg of L-malic acid, added 60 mL of n-propanol to dissolve the solution; The acid solution was added dropwise to the free base, then added 500 mL of n-heptane, stirred at room temperature to precipitate crystals, after 3 days, filtrated, and then vacuum dried at room temperature for 22 hours to obtain 2.08 g ozanimod hemi-L-malate Form 1; 89% yield.

### Example 99

Took 20 mg ozanimod free base of Preparation Example 1, added 1.5 mL of n-propanol, applied ultrasonical force to dissolve it; weighed 3.58 mg of L-malic acid, add 0.6 mL of n-propanol, applied ultrasonical force to dissolve it; the acid solution was added dropwise to the free base, then added 6 mL of n-heptane, stirred at room temperature to precipitate crystals, after 4 days, filtrated, and then vacuum dried at room temperature for 24 hours to obtain 18.9 mg ozanimod hemi- L-malate Form 1; 81% yield.

### Example 100

Took 20 mg ozanimod free base of Preparation Example 1, added 5 mL of acetone, applied ultrasonical force to dissolve it; weighed 3.72 mg of L-malic acid, added 0.6 mL of acetone, applied ultrasonical force to dissolve it; the acid solution was added drop-wisely to the free base, then added 6 mL of ethyl acetate, stirred at room temperature to precipitate crystals, after 7 days, filtrated, and then vacuum dried at room temperature for 48 hours to obtain 19.6 mg ozanimod hemi-L-malate Form 1; 85% yield.

### Example 101

Took 20 mg ozanimod free base of Preparation Example 1, added 1 mL of a mixed solvent of n-propanol and acetone (1:1 v/v), applied ultrasonical force to dissolve it; weighed 5.33 mg of L-malic acid, added 0.6 mL of a mixed solvent of n-propanol and acetone (1:1 v/v), applied ultrasonical force to dissolve it; the acid solution was added drop-wisely to the free base, then added 6 mL methyl tert-butyl ether, stirred at 10° C. to precipitate crystals, after 1 day, filtrated, and then vacuum dried at room temperature for 16 hours to obtain 16.9 mg ozanimod hemi-L-malate Form 1; 73% yield.

### Example 102

Ozanimod hemi-L-malate Form 1 can also be obtained by replacing solvents, stirring temperature and stirring time in Examples 98 to 101 with the following table.

| | | | | Stirring | Stirring | |
|---|---|---|---|---|---|---|
| Experiment | Co-solvent 1 | Co-solvent 2 | Solvent 3 | temp. | time | Yield |
| No. | (ozanimod) | (L-Malate) | (antisolvent) | (° C.) | (day) | (%) |
| Experiment 1 | butanone | ethanol | isopropyl | 10 | 5 | 68 |
| | | | acetate | | | |
| Experiment 2 | isopropanol | acetone | MTBE | 15 | 2 | 86 |
| Experiment 3 | n-propanol | methanol | ethyl acetate | 25 | 1 | 85 |
| Experiment 4 | Butanone | Butanone | n-heptane | RT | 3 | 79 |
| Experiment 5 | methanol/ | ethanol | isopropyl | 20 | 7 | 76 |
| | acetone | | ether | | | |
| Experiment 6 | 2-butanol | acetone | hexane | 35 | 2 | 76 |

The 1H-NMR data, XRPD patterns, DSC thermograms, TGA thermograms, IR spectra of the samples prepared in Examples 94 to 102 are the same as or similar to that of the sample prepared in Example 93, indicating the salts and crystalline forms obtained in Examples 94 to 102 are the same as that of Example 93.

### Examples 103-122

Referring to Examples 93 to 102, the L-malic acid was sequentially replaced with the same mole of sulfuric acid and fumaric acid, respectively, to obtain ozanimod hemi-sulfate and ozanimod hemi-fumarate, respectively.

### Example 123

The stoichiometry of the crystalline forms of ozanimod addition salts in Examples 93 to 122 is confirmed by ¹H-NMR or ion chromatography and some results are shown in the following table.

| | | | Salt molar |
|---|---|---|---|
| Ozanimod | | | ratio |
| salts | Characterization | Characterization data | (base:acid) |
| Hemi-L-malate | ¹H-NMR | ¹HNMR(500 MHz, DMSO-d6)δ | 1:0.5 |
| | | 8.51(d, J = 2.3 Hz, 1H), 8.41(dd, J = 9.0, 2.3 Hz, 1H), | |
| | | 8.11(d, J = 7.7 Hz, 1H), 7.82(d, J = 7.6 Hz, 1H), | |
| | | 7.60-7.50(m, 2H), 4.98(hept, J = 6.0 Hz, 1H), | |
| | | 4.74(dd, J = 7.8, 4.8 Hz, 1H), 3.93(dd, J = 9.4, 4.4 Hz, 1H), | |
| | | 3.65(t, J = 5.2 Hz, 2H), 3.45(ddd, J = 17.5, 8.6, 6.0 Hz, 2H), | |
| | | 3.21(ddd, J = 17.3, 8.8, 5.4 Hz, 2H), 2.95(dp, J = 23.4, 6.2, 5.5 Hz, 2 | |
| | | H), 2.33(dd, J = 15.5, 4.4 Hz, 1H), 2.17(tt, J = 8.8, 4.7 Hz, 1H), | |
| | | 1.39(d, J = 6.1 Hz, 6H) | |
| Hemi-sulfate | Ion chromatography | The sulfate ion content of the ozanimod hemi-sulfate of the | 1:0.5 |
| | | present invention is 18.9%. The theoretical sulfate ion | |
| | | content of ozanimod hemi-sulfate is 19.1%. The theoretical | |
| | | sulfate ion content of ozanimod hydrosulfate is 10.6%. | |
| Hemi-fumarate | ¹H-NMR | ¹HNMR(500 MHz, DMSO-d6)δ: 8.51(d, J = 2.2 Hz, 1H), | 1:0.5 |
| | | 8.41(dd, J = 9.0, 2.2 Hz, 1H), 8.04(d, J = 7.6 Hz, 1H), | |
| | | 7.70(d, J = 7.5 Hz, 1H), 7.56(d, J = 9.2 Hz, 1H), 7.47(t, J = 7.6 Hz, 1 | |
| | | H), 6.49(s, 1H), 4.98(p, J = 6.1 Hz, 1H), 4.48(t, J =6.4 Hz, 1H), 3.57 | |
| | | (t, J = 5.7 Hz, 3H), 3.39(ddd, J = 17.1, 8.5, 5.3 Hz, 2H), 3.14(dt, J = | |
| | | 16.9, 7.5 Hz, 2H), 2.86-2.76(m, 2H), 2.44(tt, J= 12.9, 6.8 Hz, 1H), | |
| | | 2.07-1.95(m, 1H), 1.39(d, J = 6.0 Hz, 6H) | |

### Example 124

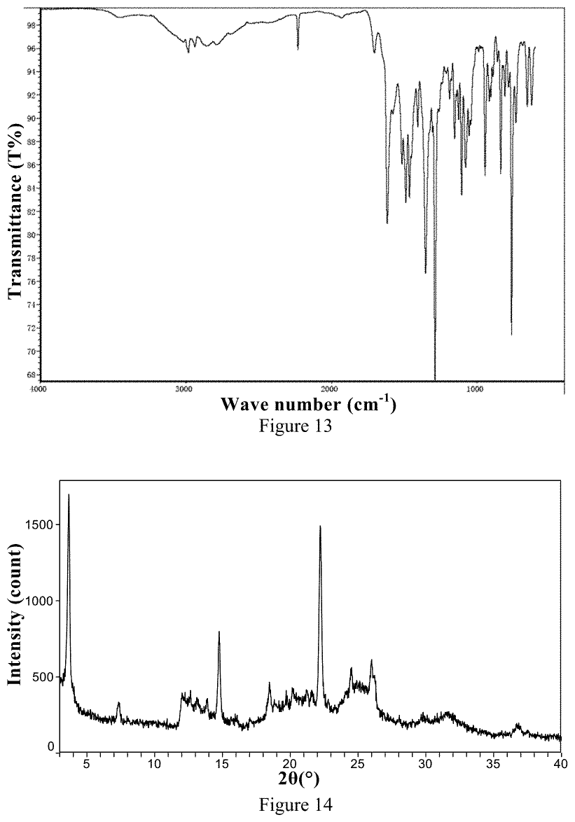

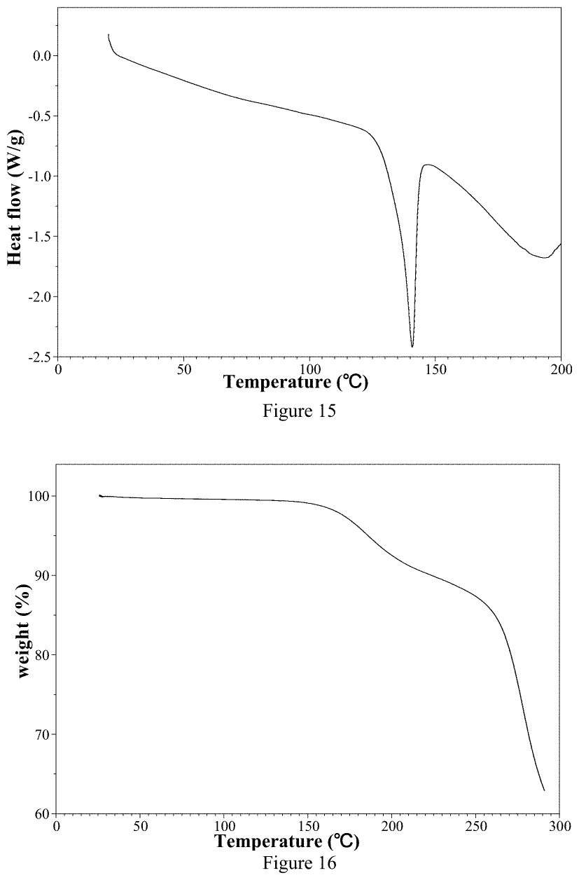

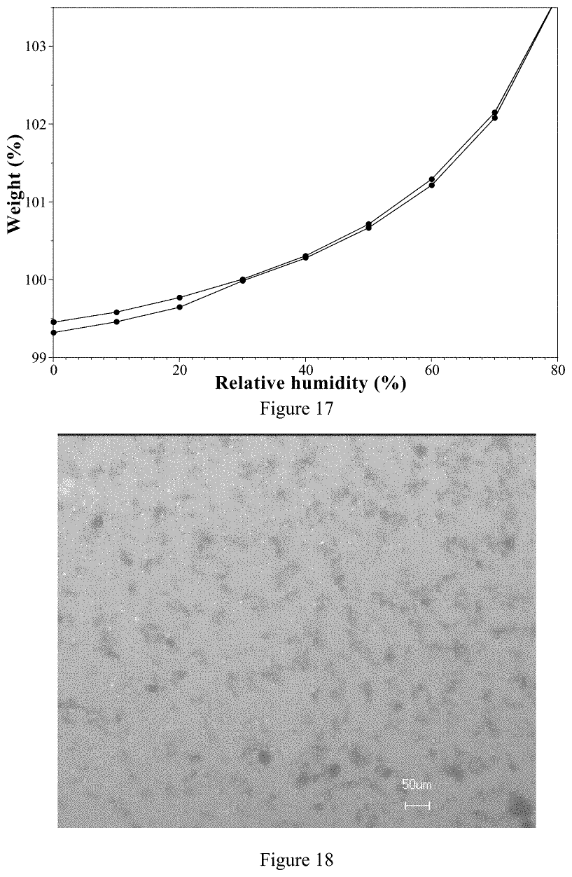

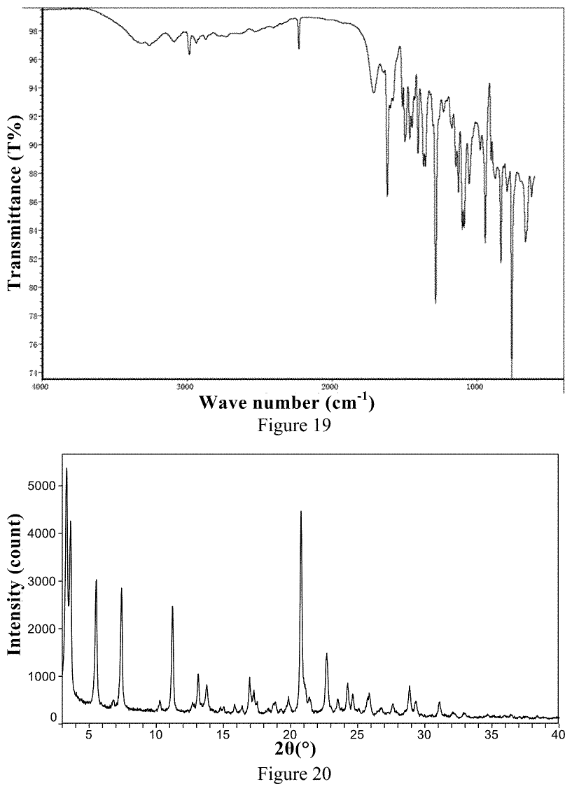

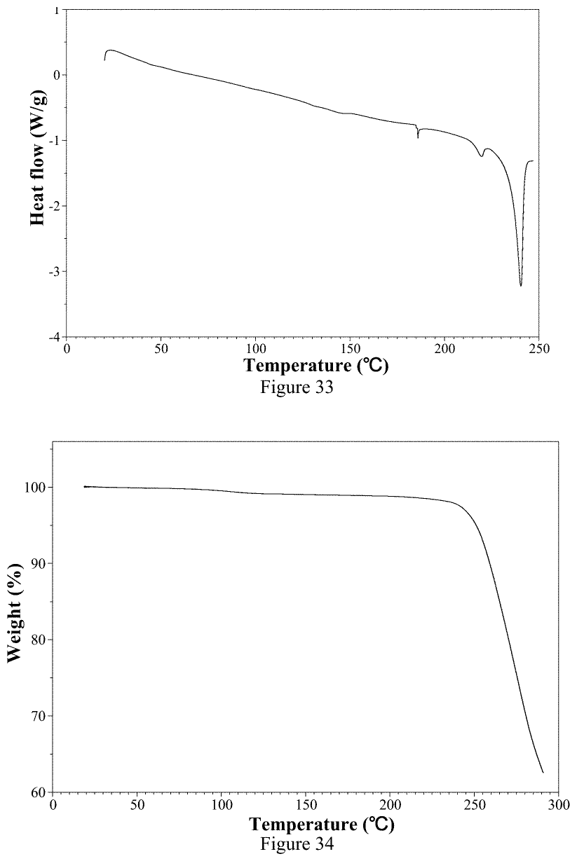

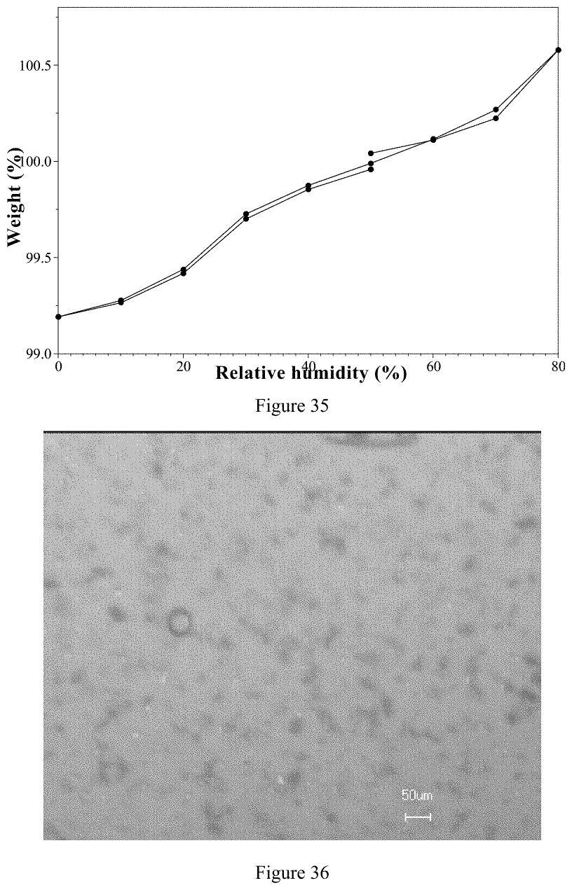

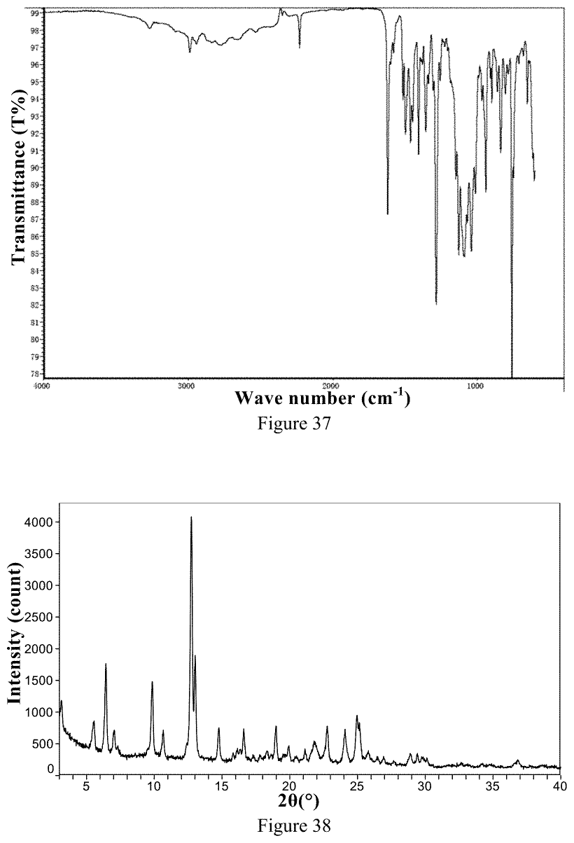

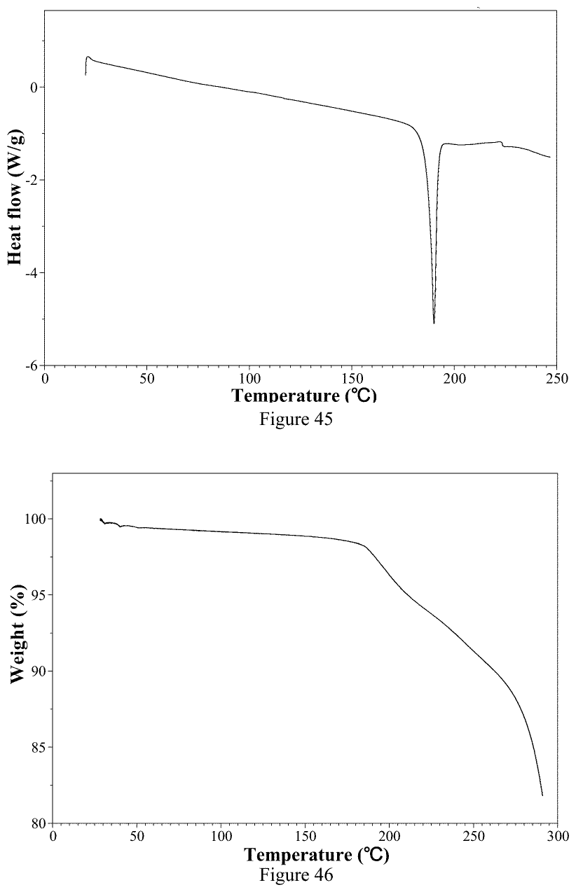

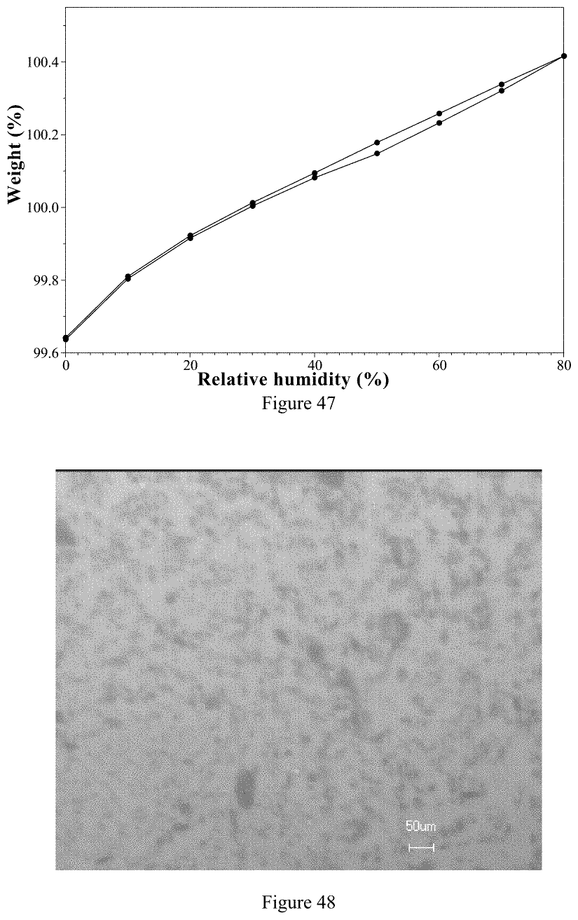

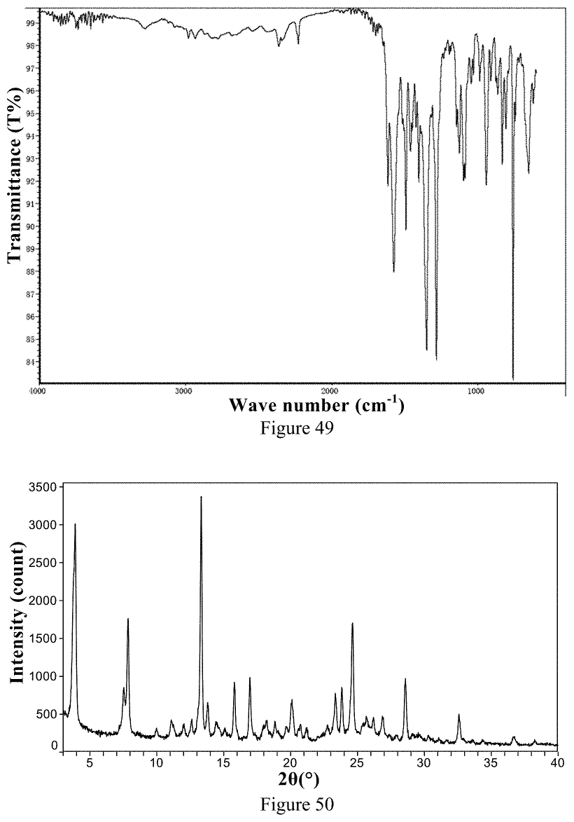

The crystalline ozanimod addition salt forms in Examples 93 to 122 were characterized by XRPD, DSC, TGA, IR and DVS, and some results are shown in the following table.

| Ozanimod salts | | | | Isothermal | | |
|---|---|---|---|---|---|---|
| and crystalline forms | XRD | DSC | TGA | adsorption | PLM | IR |
| Hemi-L-malate Form 1 | FIG. 14 | FIG. 15 | FIG. 16 | FIG. 17 | FIG. 18 | FIG. 19 |
| Sulfate Form 1 | FIG. 32 | FIG. 33 | FIG. 34 | FIG. 35 | FIG. 36 | FIG. 37 |
| Hemi-fumarate Form 1 | FIG. 44 | FIG. 45 | FIG. 46 | FIG. 47 | FIG. 48 | FIG. 49 |

### Example 125

A typical tablet was prepared by conventional tableting techniques and contained:

| | Ingredient | Weight |
|---|---|---|
| Core | ozanimod besylate Form 1 | 1.4 mg |
| | colloidal silicon dioxide | 1.0 mg |
| | microcrystalline cellulose | 56 mg |
| | modified cellulose gum | 2.0 mg |
| | magnesium stearate | adequate amount |
| Coating | HPMC | 3 mg |
| | Mywacett 9~40 T approx. | 0.3 mg |

*Acylated monoglyceride used as a plasticizer for film coating.

### Examples 126-136

Tablet: the ozanimod besylate Form 1 of Example 125 was replaced with the ozanimod citrate Form 1, ozanimod Hemi-L-malate Form 1, ozanimod dihydrogen phosphate Form 1, ozanimod hydrosulfate Form 1, ozanimod hemi-sulfate Form 1, ozanimod L-tartrate Form 1, ozanimod hemi-Fumarate form 1, ozanimod fumarate Form 1, ozanimod maleate Form 1, ozanimod hydrobromide Form 1 and ozanimod mesylate Form 1, respectively. The molar amount of the free base content of each salt in those formulations was the same as that in formulation of ozanimod besylate Form 1 of Example 125. Other components in those formulations and procedures of tableting were the same as those of Example 125.

### Example 137

A typical capsule for oral administration contained ozanimod besylate Form 1 Form 1 (1348 mg), lactose (77 mg) and magnesium stearate (15 mg). The mixture was passed through a 60 mesh sieve and packed into a No. 1 gelatin capsule.

### Example 138-148

Capsule: ozanimod besylate Form 1 of Example 137 was replaced by the ozanimod citrate Form 1, ozanimod hemi-L-malate form 1, ozanimod dihydrogen phosphate Form 1, ozanimod hydrosulfate Form 1, ozanimod hemi-sulfate Form 1, ozanimod L-tartrate Form 1, ozanimod hemi-Fumarate Form 1, ozanimod fumarate Form 1, ozanimod maleate Form 1, ozanimod hydrobromide Form 1 and ozanimod mesylate Form 1, respectively. The molar amount of the free base in these formulations was the same as that in the formulation of ozanimod besylate Form 1 of Example 137. Other components in these formulations and procedures of capsulation were the same as those of Example 137.

### Example 149

A typical injectable preparation is made by aseptically placing 348 mg ozanimod besylate Form 1 into a vial, aseptically freeze-dried and sealed. For use, the contents of the vial are mixed with 2 mL of sterile saline to produce an injectable preparation.

### Example 150-160

Injection: the ozanimod besylate Form 1 of Example 149 was replaced with the ozanimod citrate Form 1, ozanimod hemi-L-malate Form 1, ozanimod dihydrogen phosphate Form 1, ozanimod hydrosulfate Form 1, ozanimod hemi-sulfate Form 1, ozanimod L-tartrate Form 1, ozanimod hemi-fumarate Form 1, ozanimod fumarate Form 1, ozanimod maleate Form 1, ozanimod hydrobromide Form 1 and ozanimod mesylate Form 1, respectively. The molar amount of free base in these formulations was the same as that in the formulation of ozanimod besylate Form 1 of Example 149. Other components in these formulations and procedures of injection preparation were the same as those of Example 149.

### Comparative Example 1

The water solubility of crystalline forms of ozanimod salts of Examples 1 to 124 and ozanimod hydrochloride of Preparation Example 2 were tested.

The procedures are detailed as follows:

| Ozanimod salts | 25° C. water solubility | 37° C. water solubility |
|---|---|---|
| and crystalline forms | (mg/mL) | (mg/mL) |
| Ozanimod hydrochloride | 0.61 | 2.45 |
| form | | |
| Hemi-L-malate Form 1 | 10.55 | 22.62 |
| Dihydrogen phosphate | 2.54 | 7.71 |
| Form 1 | | |
| Fumarate Form 1 | 2.34 | 4.55 |
| Maleate Form 1 | 1.20 | 5.29 |
| Hydrobromide Form 1 | 0.78 | 3.49 |
| Mesylate Form 1 | 17.60 | 53.82 |

According to the above table the water solubility at 25° C. of the crystalline ozanimod hydrochloride salt form of Preparation Example 2 was 0.61 mg/mL. The water solubility of ozanimod hemi-L-malate Form 1 of the present invention was 10.55 mg/mL, the water solubility of ozanimod dihydrogen phosphate Form 1 of the present invention was 2.54 mg/mL, the water solubility of ozanimod fumarate Form 1 of the present invention was 2.34 mg/mL, the water solubility of ozanimod maleate Form 1 of the present invention was 1.20 mg/mL, the water solubility of ozanimod hydrobromide Form 1 of the present invention was 0.78 mg/mL, the water solubility of ozanimod mesylate Form 1 of the present invention was 17.60 mg/mL. Since ozanimod is a class II drug in the BCS classification, solubility is the most critical factor affecting the efficacy of the drug. Compared with the crystalline form of ozanimod hydrochloride, ozanimod hemi-L-malate Form 1, ozanimod dihydrogen phosphate Form 1, ozanimod fumarate Form 1, ozanimod maleate Form 1, ozanimod hydrobromide Form 1, and ozanimod mesylate Form 1 in the present invention have a significant advantage in solubility, which can achieve the desired blood concentration quicker and improve the therapeutic effect of the drug.

### Comparative Example 2

The phase stability of crystalline forms of ozanimod salts of Examples 1 to 124 and ozanimod hydrochloride crystalline form of Preparation Example 2 were tested in aqueous system.

The results are detailed as follows:

| | | Slurry for 5 days |
|---|---|---|
| Ozanimod addition salts and | Slurry for 5 | in ethanol/water |
| crystalline forms | days in water | (1:1) |
| Ozanimod hydrochloride form | Amorphous | Weak crystallinity |
| Ozanimod besylate Form 1 | Crystalline | Crystalline |
| Ozanimod citrate Form 1 | Crystalline | Crystalline |
| Ozanimod hemi-L-malate | Crystalline | Crystalline |
| Form 1 | | |
| Ozanimod dihydrogen | Crystalline | Crystalline |
| phosphate Form 1 | | |
| Ozanimod hydrosulfate Form 1 | Crystalline | Crystalline |
| Ozanimod hemisulfate Form 1 | Crystalline | Crystalline |
| Ozanimod L-tartrate Form 1 | Crystalline | Crystalline |
| Ozanimod hemi-fumarate | Crystalline | Crystalline |
| Form 1 | | |
| Ozanimod fumarate Form 1 | Crystalline | Crystalline |
| Ozanimod maleate Form 1 | Crystalline | Crystalline |
| Ozanimod hydrobromide | Crystalline | Crystalline |
| Form 1 | | |
| Ozanimod mesylate Form 1 | Crystalline | Crystalline |

According to the above table, compared with the ozanimod hydrochloride salt form of Preparation Example 2, the ozanimod besylate Form 1, ozanimod citrate Form 1, ozanimod hemi-L-malate Form 1, ozanimod dihydrogen phosphate Form 1, ozanimod hydrosulfate Form 1, ozanimod hemi-sulfate Form 1, ozanimod L-tartrate Form 1, ozanimod hemi-fumarate Form 1, ozanimod hemi-fumarate Form 1, ozanimod fumarate Form 1, ozanimod maleate Form 1, ozanimod hydrobromide Form 1 and ozanimod mesylate Form 1 of the present invention have better phase stability, and can better ensure that the pharmaceutical active ingredient and formulations containing ozanimod salt crystals to avoid or reduce the quality, safety and stability issues in manufacture and/or storage of the drug.

### Comparative Example 3

The HPLC purity of crystalline forms of ozanimod salts of Examples 1 to 124 and ozanimod hydrochloride of Preparation Example 2 were tested.

The results are detailed as follows:

| Ozanimod addition salts and | Purity | Yield |
|---|---|---|
| crystalline forms | (%) | (%) |
| Ozanimod Hydrochloride | 86.43 | 56 |
| Form (Before recrystallization) | | |
| Ozanimod Hydrochloride | 96.75 | |
| Form (After recrystallization) | | |
| Ozanimod besylate Form 1 | 99.12 | 94 |
| Ozanimod citrate Form 1 | 99.25 | 92 |
| Ozanimod hemi-L-malate | | |
| Form 1 | 99.17 | 92 |
| Ozanimod phosphate | | |
| dihydrogen Form 1 | 98.88 | 88 |
| Ozanimod hydrosulfate Form 1 | 98.92 | 85 |
| Ozanimod hemisulfate Form 1 | 98.79 | 87 |
| Ozanimod L-tartrate Form 1 | 99.23 | 92 |
| Ozanimod hemi-fumarate | 99.19 | 93 |
| Form 1 | | |
| Ozanimod fumarate Form 1 | 99.46 | 94 |
| Ozanimod maleate Form 1 | 99.52 | 93 |
| Ozanimod hydrobromide | 98.55 | 87 |
| Form 1 | | |
| Ozanimod mesylate Form 1 | 98.95 | 85 |

Referencing the methods described in examples [0399] in patent CN102762100B, the solvent selected for the preparation of the ozanimod hydrochloride was dioxane and diethyl ether, and the purity was relatively low, only 86.43%, and contained many impurities. The recrystallization solvent was methanol, and after recrystallization the purity of ozanimod hydrochloride was improved to 96.75%. The crystalline ozanimod addition salts of the present invention were able to achieve higher chemical purity by salt formation, for example, the purity of the crystalline ozanimod inorganic acid addition salt forms were all higher than 98.5%, and the purity of the crystalline ozanimod organic acid addition salt forms were all higher than 99.00%. In addition, the preparation of the ozanimod hydrochloride was obtained by two steps of salt formation and recrystallization, and the yield was low, only 56.0%. The crystalline forms of the ozanimod addition salts of the present invention was obtained by one-step ozanimod free base and acid addition reaction, and the yields were all higher than 85.0%, and the yield of the same organic acid addition salts was higher than 90.0%. The preparation method of the ozanimod addition salt crystalline forms of the present invention has the following advantages: simple preparation method, robust, high yield, high purity of the product, and can reduce production costs and increase production yield in mass production.

All of the patents, patent application publications, patent applications and non-patent publications cited in this specification are incorporated into this application by reference in their entireties.

The described above are only specific embodiments for illustrating the present invention, but without limiting it to that. Any changes or alternations, without creative work, made by those skilled in the art within the technical scope disclosed by the present invention, should fall within the scope of the present invention. Therefore, the scope of protection of the present invention shall be subject to the scope of protection defined in the claims.

1. A crystalline form of an ozanimod addition salt with ozanimod having structure shown in formula (A) below, 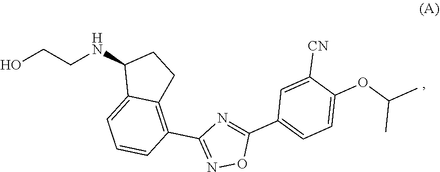 [SMILES: CC(C)Oc1ccc(-c2nc(-c3cccc4c3CC[C@@H]4NCCO)no2)cc1C#N]
wherein the crystalline form of the ozanimod addition salt is a crystalline ozanimod mono-acid salt or a crystalline ozanimod hemi-acid salt.

2. The crystalline form of the ozanimod addition salt according to claim 1, wherein, the crystalline form of the addition salt is selected from the group consisting of ozanimod besylate Form 1, ozanimod citrate Form 1, ozanimod hemi-L-malate Form 1, ozanimod dihydrogen phosphate Form 1, ozanimod hydrosulfate Form 1, ozanimod hemi-sulfate Form 1, ozanimod L-tartrate Form 1, ozanimod hemi-fumarate Form 1, ozanimod fumarate Form 1, ozanimod maleate Form 1, ozanimod hydrobromide Form 1, and ozanimod mesylate Form 1.

3. The crystalline form of the ozanimod addition salt according to any one of claims 1 to 2 is substantially crystalline solid, preferably an anhydrate, a hydrate or a non-solvate.

4. The crystalline form of the ozanimod addition salt according to claim 1 or 2, wherein, the crystalline form of the ozanimod addition saltform of the ozanimod addition salt is ozanimod besylate Form 1, wherein the X-ray powder diffraction pattern of the ozanimod besylate Form 1, expressed as 2θ angles, has the following characteristic peaks: 5.7°±0.2°, 9.1°±0.2°, 13.9°±0.2° and 14.7°±0.2°.

5. The crystalline form of the ozanimod addition salt according to claim 4, wherein the X-ray powder diffraction pattern of the ozanimod besylate Form 1, expressed as 2θ angles, further has one or more of the following characteristic peaks: 6.9°±0.2°, 11.4°±0.2°, 18.8°±0.2° and 21.6°±0.2°.

6. The crystalline form of the ozanimod addition salt form according to claim 5, wherein the X-ray powder diffraction pattern of the ozanimod besylate Form 1, expressed as 2θ angles, further has one or more of the following characteristic peaks: 23.0°±0.2°, 23.3°±0.2°, 25.1°±0.2° and 26.3°±0.2°.

7. The crystalline form of the ozanimod addition salt according to any one of claims 4 to 6, wherein the Fourier transform infrared spectrum of ozanimod besylate Form 1 has characteristic peaks at wave numbers of 1612 cm⁻¹±2 cm⁻¹, 1489 cm⁻¹±2 cm⁻¹, 1284 cm⁻¹±2 cm⁻¹, 1230 cm⁻¹±2 cm⁻¹, 1158 cm⁻¹±2 cm⁻¹, 1123 cm⁻¹±2 cm⁻¹, 1102 cm⁻¹±2 cm⁻¹, 1029 cm⁻¹±2 cm⁻¹, 1014cm⁻¹±2 cm⁻¹, 759 cm⁻¹±2 cm⁻¹, 727 cm⁻¹±2 cm⁻¹ and 614 cm⁻¹±2 cm⁻¹.

8. The crystalline form of the ozanimod addition salt according to claim 2, wherein the crystalline form of addition salt is ozanimod citrate Form 1, the X-ray powder diffraction pattern of the ozanimod citrate Form 1, expressed as 2θ angles, has the following characteristic peaks: 4.4°±0.2°, 14.0°±0.2°, 20.9°±0.2° and 24.9°±0.2°.

9. The crystalline form of the ozanimod addition salt according to claim 8, wherein the X-ray powder diffraction pattern of the ozanimod citrate Form 1, expressed as 2θ angles, further has one or more of the following characteristic peaks: 12.5°±0.2°, 13.5°±0.2°, 14.3°±0.2° and 15.9°±0.2°.

10. The crystalline form of the ozanimod addition salt form according to claim 9, wherein the X-ray powder diffraction pattern of the ozanimod citrate Form 1, expressed as 2θ angles, has one or more of the following characteristic peaks: 20.6°±0.2°, 22.7°±0.2°, 24.5°±0.2° and 29.2°±0.2°.

11. The crystalline form of the ozanimod addition salt according to any one of claims 8 to 10, wherein the Fourier transform infrared spectrum of the ozanimod citrate Form 1 has characteristic peaks at wave numbers of 1617 cm⁻¹±2 cm⁻¹, 1516 cm⁻¹±2 cm⁻¹, 1489 cm⁻¹±2 cm⁻¹, 1464 cm⁻¹±2 cm⁻¹, 1353 cm⁻¹±2 cm⁻¹, 1288 cm⁻¹±2 cm⁻¹, 1106 cm⁻¹±2 cm⁻¹, 1079 cm⁻¹±2 cm⁻¹, 945 cm⁻¹±2 cm⁻¹, 837 cm⁻¹±2 cm⁻¹ and 762 cm⁻¹±2 cm⁻¹.

12. The crystalline form of the ozanimod addition salt according to claim 2, wherein the crystalline form of the ozanimod addition salt is ozanimod hemi-L-malate Form 1, the X-ray powder diffraction pattern of the ozanimod hemi-L-malate Form 1, expressed as 2θ angles, has the following characteristic peaks: 3.7°±0.2°, 14.8°±0.2°, 18.5°±0.2° and 22.2°±0.2°.

13. The crystalline form of the ozanimod addition salt according to claim 12, wherein the X-ray powder diffraction pattern of the ozanimod hemi-L-malate Form 1, expressed as 2θ angles, further has one or more of the following characteristic peaks: 7.3°±0.2°, 12.0°±0.2°, 24.5°±0.2° and b 26.0°±0.2°.

14. The crystalline form of the ozanimod addition salt according to claim 13, wherein the X-ray powder diffraction pattern of the ozanimod hemi-L-malate Form 1, expressed as 2θ angles, further has one or more of the following characteristic peaks: 12.6°±0.2°, 13.9°±0.2°, 19.7°±0.2° and 20.1°±0.2°.

15. The crystalline form of the ozanimod addition salt according to any one of claims 12 to 14, wherein the Fourier transform infrared spectrum of the ozanimod hemi-L-malate Form 1 has characteristic peaks at wave numbers of 1710 cm⁻¹±2 cm⁻¹, 1618 cm⁻¹±2 cm⁻¹, 1496 cm⁻¹±2 cm⁻¹, 1354 cm⁻¹±2 cm⁻¹, 1284 cm⁻¹±2 cm⁻¹, 1100 cm⁻¹±2 cm⁻¹, 942 cm⁻¹±2 cm⁻¹, 833 cm⁻¹±2 cm⁻¹, 758 cm⁻¹±2 cm⁻¹ and 663 cm⁻¹±2 cm⁻¹.

16. The crystalline ozanimod addition salt form according to claim 2, wherein the crystalline addition salt form is ozanimod dihydrogen phosphate Form 1, the X-ray powder diffraction pattern of the ozanimod dihydrogen phosphate Form 1, expressed as 2θ angles, has the following characteristic peaks: 3.3°±0.2°, 5.5°±0.2°, 11.2°±0.2° and 20.8°±0.2°.

17. The crystalline ozanimod addition salt form according to claim 16, wherein the X-ray powder diffraction pattern of the ozanimod dihydrogen phosphate Form 1, expressed as 2θ angles, has one or more of the following characteristic peaks: 3.6°±0.2°, 7.4°±0.2°, 13.1°±0.2° and 22.7°±0.2°.

18. The crystalline ozanimod addition salt form according to claim 17, wherein the X-ray powder diffraction pattern of the ozanimod dihydrogen phosphate Form 1, expressed as 2θ angles, further has one or more of the following characteristic peaks: 13.8°±0.2°, 17.0°±0.2°, 24.3°±0.2° and 28.9°±0.2°.

19. The crystalline ozanimod addition salt form according to any one of claims 16 to 18, wherein the Fourier transform infrared spectrum of ozanimod dihydrogen phosphate Form 1 has characteristic peaks at wave numbers of 1618 cm⁻¹±2 cm⁻¹, 1490 cm⁻¹±2 cm⁻¹, 1464 cm⁻¹±2 cm⁻¹, 1354 cm⁻¹±2 cm⁻¹, 1288 cm⁻¹±2 cm⁻¹, 1103 cm⁻¹±2 cm⁻¹, 1006 cm⁻¹±2 cm⁻¹, 957 cm⁻¹±2 cm⁻¹, 835 cm⁻¹±2 cm⁻¹ and 762 cm⁻¹±2 cm⁻¹.

20. The crystalline ozanimod addition salt form according to claim 2, wherein the crystalline addition salt form is ozanimod hydrosulfate Form 1, the X-ray powder diffraction pattern of the ozanimod hydrosulfate Form 1, expressed as 2θ angles, has the following characteristic peaks: 4.1°±0.2°, 8.3°±0.2°, 11.1°±0.2° and 16.8°±0.2°.

21. The crystalline ozanimod addition salt form according to claim 20, wherein the X-ray powder diffraction pattern of the ozanimod hydrosulfate Form 1, expressed as 2θ angles, further has one or more of the following characteristic peaks: 14.6°±0.2°, 18.5°±0.2°, 21.3°±0.2° and 22.8°±0.2°.

22. The crystalline ozanimod addition salt form according to claim 21, wherein the X-ray powder diffraction pattern of the ozanimod hydrosulfate Form 1, expressed as 2θ angles, further has one or more of the following characteristic peaks: 17.0°±0.2°, 22.4°±0.2°, 24.7°±0.2° and 25.8°±0.2°.

23. The crystalline ozanimod addition salt form according to any one of claims 20 to 22, wherein the Fourier transform infrared spectrum of ozanimod hydrosulfate Form 1 has characteristic peaks at wave numbers of 1614 cm⁻¹±2 cm⁻¹, 1488 cm⁻¹±2 cm⁻¹, 1461 cm⁻¹±2 cm⁻¹, 1287 cm⁻¹±2 cm⁻¹, 1179 cm⁻¹±2 cm⁻¹, 1155 cm⁻¹±2 cm⁻¹, 1051 cm⁻¹±2 cm⁻¹, 867 cm⁻¹±2 cm⁻¹ and 759 cm⁻¹±2 cm⁻¹.

24. The crystalline ozanimod addition salt form according to claim 2, wherein the crystalline addition salt form is hemi-sulfate Form 1, the X-ray powder diffraction pattern of the ozanimod hemi-sulfate Form 1, expressed as 2θ angles, has the following characteristic peaks:3.8°±0.2°, 11.6°±0.2°, 13.3°±0.2° and 19.5°±0.2°.

25. The crystalline ozanimod addition salt form according to claim 24, wherein the X-ray powder diffraction pattern of the ozanimod hemi-sulfate Form 1, expressed as 2θ angles, has one or more of the following characteristic peaks:9.9°±0.2°, 15.3°±0.2°, 22.1°±0.2° and 24.6°±0.2°.

26. The crystalline ozanimod addition salt form according to claim 25, wherein the X-ray powder diffraction pattern of the ozanimod hemi-sulfate Form 1, expressed as 2θ angles, further has one or more of the following characteristic peaks: 15.7°±0.2°, 20.1°±0.2°, 25.3°±0.2° and 27.9°±0.2°.

27. The crystalline ozanimod addition salt form according to any one of claims 24 to 26, wherein the Fourier transform infrared spectrum of ozanimod hemi-sulfate Form 1 has characteristic peaks at wave numbers of 1620 cm⁻¹±2 cm⁻¹, 1462 cm⁻¹±2 cm⁻¹, 1406 cm⁻¹±2 cm⁻¹, 1284 cm⁻¹±2 cm⁻¹, 1128 cm⁻¹±2 cm⁻¹, 1090 cm⁻¹±2 cm⁻¹, 1041 cm⁻¹±2 cm⁻¹, 1013 cm⁻¹±2 cm⁻¹, 941 cm⁻¹±2 cm⁻¹, 838 cm⁻¹±2 cm⁻¹ and 761 cm⁻¹±2 cm⁻¹.

28. The crystalline ozanimod addition salt form according to claim 2, wherein the addition salt crystalline form is ozanimod L-tartrate Form 1, the X-ray powder diffraction pattern of the ozanimod L-tartrate Form 1, expressed as 2θ angles, further has the following characteristic peaks: 6.4°±0.2°, 9.9°±0.2°, 12.7°±0.2° and 22.8°±0.2°.

29. The crystalline ozanimod addition salt form according to claim 28, wherein the X-ray powder diffraction pattern of the ozanimod L-tartrate Form 1, expressed as 2θ angles, further has one or more of the following characteristic peaks: 3.1°±0.2°, 5.5°±0.2°, 10.6°±0.2° and 14.8°±0.2°.

30. The crystalline ozanimod addition salt form according to claim 29, wherein the X-ray powder diffraction pattern of the ozanimod L-tartrate Form 1, expressed as 2θ angles, has one or more of the following characteristic peaks: 7.0°±0.2°, 13.0°±0.2°, 16.6°±0.2° and 19.0°±0.2°.

31. The crystalline ozanimod addition salt form according to any one of claims 28 to 30, wherein the Fourier transform infrared spectrum of ozanimod L-tartrate Form 1 has characteristic peaks at wave numbers of 1610 cm⁻¹±2 cm⁻¹, 1569 cm⁻¹±2 cm⁻¹, 1486 cm⁻¹±2 cm⁻¹, 1460 cm⁻¹±2cm⁻¹, 1362 cm⁻¹±2 cm⁻¹, 1280 cm⁻¹±2 cm⁻¹, 1155 cm⁻¹±2 cm⁻¹, 1104 cm⁻¹±2 cm⁻¹, 1061 cm⁻¹±2 cm⁻¹, 942 cm⁻¹±2cm⁻¹ and 759 cm⁻¹±2 cm⁻¹.

32. The crystalline ozanimod addition salt form according to claim 2, wherein the crystalline addition salt form is ozanimod hemi-fumarate Form 1, the X-ray powder diffraction pattern of the ozanimod hemi-fumarate Form 1, expressed as 2θ angles, has the following characteristic peaks:6.3°±0.2°, 9.0°±0.2°, 12.6°±0.2° and 13.7°±0.2°.

33. The crystalline ozanimod addition salt form according to claim 32, wherein the X-ray powder diffraction pattern of the ozanimod hemi-fumarate Form 1, expressed as 2θ angles, further has one or more of the following characteristic peaks: 12.9°±0.2°, 14.5°±0.2°, 17.3°±0.2° and 21.5°±0.2°.

34. The crystalline ozanimod addition salt form according to claim 33, wherein the X-ray powder diffraction pattern of the ozanimod hemi-fumarate Form 1, expressed as 2θ angles, further has one or more of the following characteristic peaks: 8.6°±0.2°, 21.0°±0.2°, 22.8°±0.2° and 25.7°±0.2°.

35. The crystalline ozanimod addition salt form according to any one of claims 32 to 34, wherein the Fourier transform infrared spectrum of ozanimod hemi-fumarate Form 1 has characteristic peaks at wave numbers of 1615 cm⁻¹±2 cm⁻¹, 1576 cm⁻¹±2 cm⁻¹, 1493 cm⁻¹±2 cm⁻¹, 1405 cm⁻¹±2 cm⁻¹, 1351 cm⁻¹±2 cm⁻¹, 1284 cm⁻¹±2 cm⁻¹, 1099 cm⁻¹±2 cm⁻¹, 944 cm⁻¹±2 cm⁻¹, 833 cm⁻¹±2 cm⁻¹, 760 cm⁻¹±2 cm⁻¹ and 652 cm⁻¹±2cm⁻¹.

36. The crystalline ozanimod addition salt form according to claim 2, wherein the crystalline addition salt form is ozanimod fumarate Form 1, the X-ray powder diffraction pattern of the ozanimod fumarate Form 1, expressed as 2θ angles, has the following characteristic peaks: 3.9°±0.2°, 7.9°±0.2°, 13.3°±0.2° and 17.0°±0.2°.

37. The crystalline ozanimod addition salt form according to claim 36, wherein the X-ray powder diffraction pattern of the ozanimod fumarate Form 1, expressed as 2θ angles, further has one or more of the following characteristic peaks: 7.5°±0.2°, 15.8°±0.2°, 24.6°±0.2° and 28.6°±0.2°.

38. The crystalline ozanimod addition salt form according to claim 37, wherein the X-ray powder diffraction pattern of the ozanimod fumarate Form 1, expressed as 2θ angles, further has one or more of the following characteristic peaks: 13.8°±0.2°, 20.1°±0.2°, 23.3°±0.2° and 23.8°±0.2°

39. The crystalline ozanimod addition salt form according to any one of claims 36 to 38, wherein the Fourier transform infrared spectrum of ozanimod fumarate Form 1 has characteristic peaks at wave numbers of 1701 cm⁻¹±2 cm⁻¹, 1614 cm⁻¹±2 cm⁻¹, 1484 cm⁻¹±2 cm⁻¹, 1462 cm⁻¹±2 cm⁻¹, 1342 cm⁻¹±2 cm⁻¹, 1284 cm⁻¹±2 cm⁻¹, 1103 cm⁻¹±2 cm⁻¹, 986 cm⁻¹±2 cm⁻¹, 759 cm⁻¹±2 cm⁻¹ and 639 cm⁻¹±2 cm⁻¹.

40. The crystalline ozanimod addition salt form according to claim 2, wherein the addition salt crystalline form is ozanimod maleate Form 1, the X-ray powder diffraction pattern of the ozanimod maleate Form 1, expressed as 2θ angles, has the following characteristic peaks: 8.2°±0.2°, 11.5°±0.2°, 12.4°±0.2° and 13.6°±0.2°.

41. The crystalline ozanimod addition salt form according to claim 40, wherein the X-ray powder diffraction pattern of the ozanimod maleate Form 1, expressed as 2θ angles, further has the following one or more characteristic peaks: 5.3°±0.2°, 6.7°±0.2°, 10.2°±0.2° and 11.0°±0.2°.

42. The crystalline ozanimod addition salt form according to claim 41, wherein the X-ray powder diffraction pattern of the ozanimod maleate Form 1, expressed as 2θ angles, further has one or more of the following characteristic peaks: 14.1°±0.2°, 15.8°±0.2°, 16.4°±0.2° and 18.1°±0.2°.

43. The crystalline ozanimod addition salt form according to any one of claims 40 to 42, wherein the Fourier transform infrared spectrum of ozanimod maleate Form 1 has characteristic peaks at wave numbers of 1700 cm⁻¹±2 cm⁻¹, 1614 cm⁻¹±2 cm⁻¹, 1487 cm⁻¹±2 cm⁻¹, 1461 cm⁻¹±2 cm⁻¹, 1353 cm⁻¹±2 cm⁻¹, 1281 cm⁻¹±2 cm⁻¹, 1102 cm⁻¹±2 cm⁻¹, 1087 cm⁻¹±2 cm⁻¹, 865 cm⁻¹±2 cm⁻¹, 759 cm⁻¹±2 cm⁻¹ and 653 cm⁻¹±2 cm⁻¹.

44. The crystalline ozanimod addition salt form according to claim 2, wherein the crystalline addition salt form is ozanimod hydrobromide Form 1, the X-ray powder diffraction pattern of the ozanimod hydrobromide Form 1, expressed as 2θ angles, has the following characteristic peaks: 3.9°±0.2°, 12.1°±0.2°, 13.7°±0.2° and 20.3°±0.2°.

45. The crystalline ozanimod addition salt form according to claim 44, wherein the X-ray powder diffraction pattern of the ozanimod hydrobromide Form 1, expressed as 2θ angles, further has one or more of the following characteristic peaks: 12.9°±0.2°, 22.7°±0.2°, 24.5°±0.2° and 26.2°±0.2°.

46. The crystalline ozanimod addition salt form according to claim 45, wherein the X-ray powder diffraction pattern of the ozanimod hydrobromide Form 1, expressed as 2θ angles, further has one or more of the following characteristic peaks: 12.4°±0.2°, 19.5°±0.2°, 21.3°±0.2° and 26.8°±0.2°.

47. The crystalline ozanimod addition salt form according to any one of claims 44 to 46, wherein the Fourier transform infrared spectrum of hydrobromide Form 1 has characteristic peaks at wave numbers of 3276 cm⁻¹±2 cm⁻¹, 1620 cm⁻¹±2 cm⁻¹, 1498 cm⁻¹±2 cm⁻¹, 1443 cm⁻¹±2 cm⁻¹, 1405 cm⁻¹±2 cm⁻¹, 1353 cm⁻¹±2 cm⁻¹, 1285 cm⁻¹±2 cm⁻¹, 1099 cm⁻¹±2 cm⁻¹, 1074 cm⁻¹±2 cm⁻¹, 942 cm⁻¹±2 cm⁻¹, 837 cm⁻¹±2 cm⁻¹ and 761 cm⁻¹±2 cm⁻¹.

48. The crystalline ozanimod addition salt form according to claim 2, wherein the crystalline addition salt form is ozanimod mesylate Form 1, the X-ray powder diffraction pattern of the ozanimod mesylate Form 1, expressed as 2θ angles, has the following characteristic peaks: 11.6°±0.2°, 12.6°±0.2°, 18.2°±0.2° and 19.5°±0.2°.

49. The crystalline ozanimod addition salt form according to claim 48, wherein the X-ray powder diffraction pattern of the ozanimod mesylate Form 1, expressed as 2θ angles, further has one or more of the following characteristic peaks: 4.9°±0.2°, 7.9°±0.2°, 9.9°±0.2° and 16.8°±0.2°.

50. The crystalline ozanimod addition salt form according to claim 49, wherein the X-ray powder diffraction pattern of the ozanimod mesylate Form 1, expressed as 2θ angles, further has one or more of the following characteristic peaks: 20.1°±0.2°, 23.1°±0.2°, 23.4°±0.2°, 24.3°±0.2° and 25.0°±0.2°.

51. The crystalline ozanimod addition salt form according to any one of claims 48 to 50, wherein the Fourier transform infrared spectrum of ozanimod mesylate Form 1 has characteristic peaks at wave numbers of 1617 cm⁻¹±2 cm⁻¹, 1492 cm⁻¹±2 cm⁻¹, 1406 cm⁻¹±2 cm⁻¹, 1357 cm⁻¹±2 cm⁻¹, 1285 cm⁻¹±2 cm⁻¹, 1152 cm⁻¹±2 cm⁻¹, 1105 cm⁻¹±2 cm⁻¹, 1044 cm⁻¹±2 cm⁻¹, 940 cm⁻¹±2 cm⁻¹, 780 cm⁻¹±2 cm⁻¹ and 760 cm⁻¹±2 cm⁻¹.

52. The preparation method of crystalline ozanimod addition salt form according to any one of claims 1 to 2, wherein forming a solution of ozanimod and a solution of the corresponding acid of claim 2 in a co-solvent, respectively, and then mixing, and completing preparation of the crystalline form by the following method I or method II:
method I: Stirring the mixture, separation and drying of precipitated crystals to obtain the ozanimod mono-acid addition salt or the ozanimod hemi-acid addition salt;
method II: Adding an anti-solvent to the mixed solution, stirring and separating the precipitated crystals to obtain a crystalline form of the ozanimod mono-acid addition salt or the ozanimod hemi-acid addition salt.

53. The preparation method of the crystalline ozanimod addition salt form according to claim 52, wherein in the method I or the method II, the co-solvent is an alcohol, a ketone or a mixture thereof;
preferably, in the method I or the method II, the co-solvent is selected from the group consisting of a C1 to C4 alcohol, a C3 to C4 ketone or a mixture thereof, more preferably n-propanol, acetone or a mixture thereof;
preferably, in the method I, the concentration of ozanimod in co-solution is 0.5 to 1.05 times of the solubility of ozanimod in the selected solvent;
preferably, in the method II, the concentration of ozanimod in co-solution is 0.1 to 1.05 times of the solubility of ozanimod in the selected solvent, more preferably 0.1 to 0.4 times;
preferably, in the method II, the anti-solvent is selected from the group consisting of an ester, an ether, an alkane or a mixture thereof, more preferably ethyl acetate, methyl tert-butyl ether, n-heptane or their mixture thereof;
preferably, in the preparation of the ozanimod mono-acid salt, the molar ratio of ozanimod and acidic counter ion is 1:1.0 to1:1.5, more preferably 1:1.0 to 1:1.2;
preferably, in the preparation of the ozanimod hemi-acid salt, the molar ratio of ozanimod and acidic counter ion is 1:0.5 to 1:0.8, more preferably 1:0.5 to 1:0.6;
preferably, the stirring time is 1 to 7 days, more preferably 3 to 7 days;
preferably, the operating temperature of the preparation method is 10 to 40° C., more preferably room temperature;
preferably, the drying temperature is room temperature, and the drying time is 16 to 48 hours.

54. A pharmaceutical composition, which comprises a therapeutically and/or preventively effective amount of the active pharmaceutical ingredient selected from the crystalline ozanimod addition salt forms according to any one of claims 1 to 51, and at least one pharmaceutically acceptable carrier or additive.

55. The crystalline ozanimod addition salt form according to any one of claims 1 to 51, or the pharmaceutical composition according to claim 54 for treating and/or preventing one or more diseases or adverse conditions; the diseases or adverse conditions are associated with modulation, activation, stimulation, inhibition or antagonization of selective sphingosine-1-phosphate (S1P) receptor.

56. A treating and/or preventing method for one or more diseases or adverse conditions comprises a therapeutically and/or preventively effective amount of active pharmaceutical ingredient selected from the crystalline ozanimod addition salt forms according to any one of claims 1 to 51, or a pharmaceutical composition according to any one of claim 54 or 55; the diseases or adverse conditions are associated with modulation, activation, stimulation, inhibition or antagonization of selective sphingosine-1-phosphate (SIP) receptor; the diseases or adverse conditions include but not limited to multiple sclerosis, ulcerative colitis, arthritis, transplant rejection or acute respiratory distress syndrome.
